(19) United States
(12) Patent Application Publication
(10) Pub. No.: US 2022/0401509 A1
(43) Pub. Date: Dec. 22, 2022

(54) Composition For Improving Mental State, Production Method For Composition For Improving Mental State, And Use Of Fruit Of Eggplant For Improving Mental State

(71) Applicants: SHINSHU UNIVERSITY, Nagano (JP); ELECTRONICS DEVELOPMENT COMPUTER COLLEGE, Tokyo (JP); WELLNAS.CO.,LTD., Tokyo (JP)

(72) Inventors: Kozo NAKAMURA, Nagano (JP); Jun NISHIHIRA, Hokkaido (JP)

(21) Appl. No.: 17/776,939

(22) PCT Filed: Nov. 12, 2020
(86) PCT No.: PCT/JP2020/042298

§ 371 Date: May 13, 2022

(30) Foreign Application Priority Data
Nov. 15, 2019 (JP) 2019-207531

(51) Int. Cl. A61K 36/81; A61P 25/22

(52) U.S. Cl. CPC A61K 36/81; A61P 25/22; A61K 2236/15; A61K 2236/37; A61K 2236/51

(57) ABSTRACT

The present invention addresses the problem of providing a novel composition having the effect of improving a mental state. The present invention pertains to: a composition that is for improving a mental state, and that contains, as an active ingredient, a water-soluble component of the fruit of eggplant (Solanum melongena); and a production method that is for an eggplant-derived composition for improving a mental state, and that comprises squeezing an eggplant fruit to obtain a juice, or adding water to an eggplant fruit and squeezing the fruit to which water has been added, to obtain a juice.

### TECHNICAL FIELD

This disclosure relates to a use of eggplant fruit for psychological state improving composition, manufacturing method of psychological state improving composition, and psychological state improvement.

### BACKGROUND ART

According to a report published on World Health Day, cardiovascular disease accounts for approximately 17 million deaths per year, corresponding to almost one third of total global mortality [Non patent literature 1]. Hypertension accounts for 9.4 million deaths worldwide every year, contributing to at least 45% of deaths attributed to heart disease and 51% of those attributed to stroke. Hypertension also contributes to kidney failure, premature mortality, and disability [Non patent literature 1]. Therefore, prevention and management of hypertension are major public health challenges worldwide. Several studies have shown that lifestyle modifications, including dietary changes, physical exercise, and weight loss, can contribute to the primary prevention of hypertension and significantly reduce blood pressure (BP) in people with established hypertension [Non patent literatures 2-4]. Functional foods with antihypertensive effects are also expected to prevent and manage hypertension [Non patent literature 5]. In Japan, functional foods for people with higher than normal BP are commercially available as government-approved “Food for Specified Health Uses” or “Foods with Function Claims” with the food business operator's own responsibility.

Eggplant (Solanum melongena) is a globally popular vegetable consumed daily, and an agronomically and economically important cultivated crop. Eggplant is low in calories, carbohydrate, and protein, and rich in dietary fiber and minerals [Non patent literature 6]; thus, it is recommended for reducing caloric intake and body weight (BW) to prevent type-2 diabetes [Non patent literatures 7-9]. Our research found only two clinical trials investigating eggplant related to the cholesterol-lowering effects in hypercholesterolemic participants [Non patent literature 10] and its efficacy in reducing fat mass in overweight women [Non patent literature 11].

Acetylcholine (ACh) is well-known as a neurotransmitter and present in the nervous system of not only mammals but also almost all organisms, including those used as food for humans [Non patent literature 12]. Food materials such as eggplant, bamboo shoots (Phyllostachys bambusoides), and shiitake mushroom (Lentinus eddoes) are reported to contain ACh [Non patent literatures 12, 13]. Inventors of this disclosure have reported the isolation of ACh and lactoylcholine from lactic acid bacteria-fermented food with a hypotensive effect in spontaneously hypertensive rats (SHRs) and reported that choline esters might be responsible for the BP-lowering effect [Non patent literatures 14, 15]. Recently, the inventors presented the antihypertensive effects of orally administered eggplant powder rich in ACh in SHRs [Non patent literature 16] and suggested that ACh in eggplant suppressed sympathetic nervous activity to cause the effect. Bethanechol, an artificial cholinergic agent, stimulates the parasympathetic nervous system through muscarinic ACh receptor [Non patent literature 17] and lowers BP. Therefore, ACh in orally administered eggplant was estimated to promote parasympathetic nervous activity and then suppress sympathetic nervous activity by reciprocal innervation and lower BP. Stress and psychological state (PS) are closely related to sympathetic nervous activity [Non patent literatures 18, 19]. Psychosocial factors are also related to blood pressure and may lead to hypertension [Non patent literature 20]. Therefore, eggplant ingestion may improve stress and PSs, in addition to BP.

The present inventor has studied the antihypertensive effects and vasodilator components in fermented buckwheat (lactic acid fermented buckwheat plant) and has provided an extract composition containing quaternary alkylammonium compounds mainly composed of multiple choline esters, including at least acetylcholine and propionylcholine. Inventors also investigated the antihypertensive effects of single oral administration of purified ACh, propionylcholine, butyrylcholine, and lactoylcholine to SHR. We also found that purified ACh, propionylcholine, butyrylcholine, and lactoylcholine had an antihypertensive effect when administered orally as a single dose to SHR (Patent literatures 1, 2). Furthermore, the inventor has found that freeze-dried powders of edible plants such as eggplant and bamboo shoots and extracts of edible plants with ethanol or hydrous ethanol contain active ingredients with antihypertensive effects such as acetylcholine, which can be used for oral intake (Patent literature 3).

On the other hand, there is a report that a composition containing pressed vegetables such as tomatoes, carrots, and spinach and pressed sprouted buckwheat as active ingredients can be used as an anti-hypertensive agent (Patent literature 4), and a composition containing a water-soluble, sugar-free tomato extract can be used as an anti-hypertensive agent (Patent literature 5). However, these were not easy to prepare because they required multiple raw materials or the removal of sugar from the extracts.

### PRIOR ART LITERATURES

### Patent Literature

[Patent literature 1] WO2015147251
[Patent literature 2] JP2015189745A
[Patent literature 3] WO2018070545
[Patent literature 4] JP2008214221A
[Patent literature 5] JP2015515493A

### Non Patent Literature

[Non patent literature 1] World Health Organization. A Global Brief on Hypertension: Silent killer, Global Public Health Crisis. 2013. Available online: https://www.who.int/cardiovascular_diseases/publications/global_brief_hypertension/en/ (accessed on 29 Mar. 2018).
[Non patent literature 2] Chobanian, A. V.; Bakris, G. L.; Black HR; Cushman, W. C.; Green, L. A.; Izzo, J. L. Jr; Jones, D. W.; Materson, B. J.; Oparil, S.; Wright, J. T., Jr.; et al. The National High Blood Pressure Education Program Coordinating Committee. The seventh report of the joint national committee on prevention, detection, evaluation, and treatment of high blood pressure. JAMA 2003, 289, 2560-2571.
[Non patent literature 3] Go, A. S.; Bauman, M. A.; Coleman, King, S. M.; Fonarow, G. C.; Lawrence, W.; Williams, K. A.; Sanchez, E. An effective approach to high blood pressure control: A science advisory from the American Heart Association, the American College of Cardiology, and the Centers for Disease Control and Prevention. J. Am. Coll. Cardiol. 2014, 63, 1230-1238.
[Non patent literature 4] Williams, B.; Poulter, N. R.; Brown, M. J.; Davis, M.; McInnes, G. T.; Potter, J. F.; Sever, P. S.; McG, Thom, S. British Hypertension Society. Guidelines for management of hypertension: Report of the fourth working party of the British Hypertension Society, 2004-BHS IV. J. Hum. Hypertens. 2004, 18, 139-185.
[Non patent literature 5] Mohamed, S. Functional foods against metabolic syndrome (obesity, diabetes, hypertension and dyslipidemia) and cardiovascular disease. Trends Food Sci. Tech. 2014, 35, 114-128.
[Non patent literature 6] United States Department of Agriculture Agricultural Research Service (USDA ARS). National Nutrient Database for Standard Reference Release 28 (https://ndb.nal.usda.gov/ndb/). Available online: https://ndb.nal.usda.gov/ndb/foods/show/2962?fgcd=&manu=&lfacet=&format=&count=&max=50&offset=&sort=default&order=asc&qlookup=eggplant&ds=&qt=&qp=&qa=&qn=&q=&ing=/ (accessed on 14 Mar. 2018).
[Non patent literature 7] National Institute of Diabetes and Digestive and Kidney Diseases (NIDDK), National Institutes of Health (NIH). Nutrition Research Report 2015 & 2016. Preventing type 2 diabetes. Available online: https://www.niddk.nih.gov/health-information/diabetes/overview/preventing-type-2-diabetes/ (accessed on 29 Mar. 2018).
[Non patent literature 8] American Diabetes Association (ADA). Eating colorful food has health benefits. Available online: http://www.diabetesforecast.org/2011/aug/eating-colorful-food-has-health-benefits.html (accessed on 29 Mar. 2018).
[Non patent literature 9] American Diabetes Association (ADA). Food for your plate. Available online: http://www.diabetesforecast.org/2015/adm/diabetes-plate-method/foods-for-your-plate.html (accessed on 29 Mar. 2018).
[Non patent literature 10] Guimaraes, P. R; Galvao, A. M. P.; Batista, C. M.; Azevedo, G. S.; Oliveira, R. D.; Lamounier, R. P.; Freire, N.; Barros, A. M. D.; Sakurai, E.; Oliveira, J. P.; et al. Eggplant (Solanum melongena) infusion has a modest and transitory effect on hypercholesterolemic subjects. Braz. J. Med. Biol. Res. 2000, 33, 1027-1036.
[Non patent literature 11] Scorsatto, M.; Rosa, G.; Luiz, R. R.; Mulder, A. R. P.; Teodoro, A. J.; Oliveiram g. M. M. Effect of eggplant flour (Solanum melongena L.) associated with hypoenergetic diet on antioxidant status in overweight women-A randomised clinicaltrial. Int. J. Food Sci. Techol. 2019, 54, 2182-2189.
[Non patent literature 12] Horiuchi, Y.; Kimura, R; Kato. N.; Fujii, T.; Seki, M.; Endo, T.; Kato, T.; Kawashima, K. Evolutional study on acetylcholine expression. Life Sci. 2003, 72, 1745-1756. [Non patent literature 13] Kawashima, K. Origin of acetylcholine and expression of non-neuronal acetylcholine. Biomed. Gerontol. 2010, 34, 12-24.
[Non patent literature 14] Nakamura, K.; Naramoto, K.; Koyama, M. Blood-pressure-lowering effect of fermented buckwheat sprouts in spontaneously hypertensive rats. J. Funct. Foods 2013, 5, 406415.
[Non patent literature 15] Nakamura, K.; Okitsu, S.; Ishida, R.; Tian, S.; Igari, N.; Amano, Y. Identification of natural lactoylcholine in lacticacid bacteria-fermented food. Food Chem. 2016, 201, 185-189.
[Non patent literature 16] Yamaguchi, S.; Matsumoto, K.; Koyama, M.; Tian. S.; Watanabe, M.; Takahashi, A.; Miyatake, K.; Nakamura, K. Antihypertensive effects of orally administered eggplant (Solanum melongena) rich in acetylcholine on spontaneously hypertensive rats. Food Chem. 2019, 276, 376-382.
[Non patent literature 17] Molitor, H. A comparative study of the effects of five choline compounds used in therapeutics: Acetylcholine chloride, acetyl beta-methylcholine chloride, carbaminoyl choline, ethyl ether beta-methylcholine chloride, carbaminoyl beta-methylcholine chloride. J. Pharmacol. Exp. Ther. 1936, 58, 337-360.
[Non patent literature 18] Wilson, M. A.; Fadel, J. R. Cholinergic regulation of fear learning and extinction. J. Neurosci. Res. 2017, 95, 836-852.
[Non patent literature 19] Balkan, B.; Pogun, S. Nicotinic cholinergic system in the hypothalamus modulates the activity of the hypothalamic neuropeptides during the stress response. Curr. Neuropharmacol. 2018, 16, 371-387.
[Non patent literature 20] Steptoe, A. Psychosocial factors in the development of hypertension. Ann. Med. 2000, 32, 371-375.

### SUMMARY OF INVENTION

### Problems to be Solved

The inventor explored new materials with psychological state improvement effects, noting that if compositions with psychological state improvement effects could be obtained more effectively and more easily prepared using fewer raw materials, they could be mass-produced at low cost as health foods such as supplements and pharmaceuticals.

Therefore, the goal of the present invention is to provide a new composition with psychological state improvement properties.

### The Means to Solve the Problems

The principal invention to solve the above problem is a composition for improving psychological states, which contains the water-soluble component of eggplant (Solanum melongena) fruit as an active ingredient.

Other problems disclosed in this application and their solutions will be explained in the section on embodiments of the invention and in the drawings.

### Effects of Invention

According to the present invention, a new composition with psychological state improvement effects can be provided.

### BRIEF DESCRIPTION OF DRAWINGS

FIG. 1 shows a flowchart of a participant selection in a randomized, double-blind, placebo-controlled, parallel group comparison study to examine the effects of continuous intake of eggplant powder on BP, stress, and PS.

FIG. 2 shows a study schedule.

FIG. 3 shows the effect of continuous intake of eggplant powder on BP improvement and the changes in hospital diastolic blood pressure (DBP) for all participants (normal-high BP or grade 1 hypertension).

FIG. 4 shows the effect of continuous intake of eggplant powder on BP improvement and the changes in hospital DBP for normal-high BP participants.

FIG. 5 shows the effect of continuous intake of eggplant powder on BP improvement and the measured values in hospital systolic blood pressure (SBP) for all participants (normal-high BP or grade 1 hypertension).

FIG. 6 shows the effect of continuous intake of eggplant powder on BP improvement and the measured values in hospital SBP for grade 1 hypertension participants.

FIG. 7 shows the effect of continuous intake of eggplant powder on BP improvement and the measured values in hospital DBP for grade 1 hypertension participants.

FIG. 8 shows the effect of continuous intake of eggplant powder on BP improvement and the changes in home/morning BP for all participants (normal-high BP or grade 1 hypertension).

FIG. 9 shows the effect of continuous intake of eggplant powder on BP improvement and the changes in home/morning BP for normal-high BP participants.

FIG. 10 shows the effect of continuous intake of eggplant powder on BP improvement and the changes in home/evening DBP for normal-high BP participants.

FIG. 11 shows the effect of continuous intake of eggplant powder on PSs improvement and the changes in Friendliness for grade 1 hypertension participants.

FIG. 12 shows the effect of continuous intake of eggplant powder on PSs improvement and the changes in vigor-activity for grade 1 hypertension participants.

FIG. 13 shows the effect of continuous intake of eggplant powder on PSs improvement and measured values of confusion-bewilderment scores for all participants (normal-high BP or grade 1 hypertension).

FIG. 14 shows the effect of continuous intake of eggplant powder on PSs improvement and measured values of depression-dejection scores for normal-high BP participants.

FIG. 15 shows the effect of continuous intake of eggplant powder on PSs improvement and measured values of confusion-bewilderment scores for normal-high BP participants.

FIG. 16 shows the effect of continuous intake of eggplant powder on PSs improvement and measured values of anger-hostility scores for normal-high BP participants.

FIG. 17 shows the effect of continuous intake of eggplant powder on PSs improvement and measured values of total mood disturbance (TMD) for normal-high BP participants.

FIG. 18 is a part of Table S1.

FIG. 19 is a part of Table S1.

FIG. 20 is a part of Table S2.

FIG. 21 is a part of Table S2.

FIG. 22 is a part of Table S2.

FIG. 23 is a part of Table S3.

FIG. 24 is a part of Table S3.

FIG. 25 is a part of Table S3.

FIG. 26 is a part of Table S3.

FIG. 27 is a part of Table S4.

FIG. 28 is a part of Table S4.

FIG. 29 is a part of Table S4.

FIG. 30 is Table S5.

FIG. 31 is a part of Table S6.

FIG. 32 is a part of Table S6.

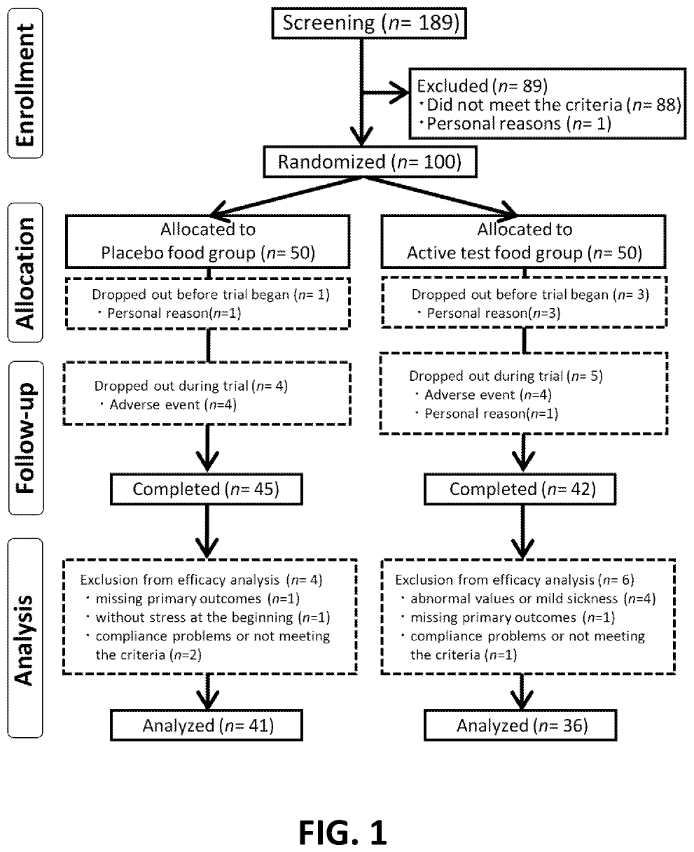

### DETAILED DESCRIPTION OF INVENTION

The contents of the embodiments of the present invention will be listed and described. The present invention has, for example, the following configuration.

### [Item 1]

A composition for improving a psychological state, which comprises a fruit-derived component of eggplant (Solanum melongena).

### [Item 2]

The composition for improving a psychological state according to item 1, which comprises a water-soluble component of the fruit of eggplant (Solanum melongena) as an active ingredient.

### [Item 3]

The composition for improving a psychological state according to item 1, which consists of a water-soluble component of the fruit of eggplant (Solanum melongena).

### [Item 4]

The composition for improving a psychological state as in one of items 1-3,

wherein it is orally ingested at a dose such that an amount of choline ester in the water-soluble component is greater than or equal to 0.5 μg/kg body weight and less than or equal to 50 mg/kg body weight.

### [Item 5]

The composition for improving a psychological state as in one of Items 1-4, which has a choline ester content of greater than or equal to 5 g and less than or equal to 50 mg and is for oral ingestion.

### [Item 6]

The composition for improving a psychological state according to item 4 or 5, wherein the choline ester is selected from any one of acetylcholine, butylcholine and propionylcholine.

### [Item 7]

The composition for improving a psychological state as in one of items 1-6, which is a dry powder.

### [Item 8]

The composition for improving a psychological state as in one of items 1-7, which is a food composition.

### [Item 9]

A method for producing a composition for improving a psychological state, which comprises squeezing an eggplant (Solanum melongena) fruit to obtain a juice.

### [Item 10]

A method for producing a composition for improving a psychological state containing an eggplant-derived component,

wherein water is added to a fruit of eggplant (Solanum melongena) and the fruit to which the water is added is squeezed to obtain a juice.

### [Item 11]

The method for producing the psychological state improving composition according to item 10, which comprises crushing the fruit before or after adding the water to the fruit.

### [Item 12]

The method for producing the composition for improving a psychological state as in one of items 9-11, which comprises heating the fruit.

### [Item 13]

The method for producing the composition for improving a psychological state as in one of items 9-12, which comprises drying the juice into a powder.

### [Item 14]

A composition for improving a psychological state produced by the production method as in one of items 9-13.

### [Item 15]

Use of eggplant (Solanum melongena) fruit to improve a psychological state.

### [Item 16]

The use of eggplant fruit according to item 15 by oral ingestion.

### [Item 17]

A composition for improving a psychological state, which comprises a water-soluble component of a food made from an agricultural product containing a high amount of choline ester as an active ingredient.

Hereinafter, the composition for improving a psychological state and the method for producing the composition for improving a psychological state according to the embodiment will be described.

### 1. Composition for Improving a Psychological State

The psychological state improving composition according to one embodiment contains a water-soluble component of eggplant fruit as an active ingredient. Further, the composition for improving a psychological state according to another embodiment may consist of a water-soluble component of eggplant fruit.

Here, the water-soluble component of the eggplant fruit could mean the water-soluble component obtained by squeezing the eggplant fruit, but it could also mean the water-soluble component in the juice obtained by adding water to the eggplant fruit and squeezing it. For example, even if the juice contains a water-insoluble component inevitably, or even if a part of the water-soluble component inevitably remains as a residue during juice extraction, the component contained in the juice corresponds to the water-soluble component.

The eggplant used in this embodiment is not particularly limited. Commonly eaten eggplants are preferred, and varieties such as Tosataka, Shintaro, Ryoma, Senshu Mizunasu, Battennasu, Koryo Salad Eggplant (also known as Binan), Higomurasaki, Oonaga Eggplant, Chikuyo, and Senryo might be used.

In the present embodiment, examples of the choline ester that can be contained in the composition for improving a psychological state include ACh, butyrylcholine, propionylcholine, and the like, and a composition containing one or more of these may be used.

In the present embodiment, when the composition for improving a psychological state is used for humans, the amount of ACh in the water-soluble component is 0.5 μg/kg body weight to 50 mg/kg body weight, preferably 2 μg/kg body weight to 20 mg/kg. It is used for oral ingestion at a dose of kg body weight, particularly preferably a dose of 5 μg/kg body weight to 1 mg/kg body weight, more preferably 8 μg/kg body weight to 100 μg/kg body weight. By orally ingesting at such a dose, a psychological state improving effect can be obtained more effectively.

In the present embodiment, the composition for improving a psychological state is for oral intake, and the choline ester content may be 5 μg or more and 50 mg or less. Further, in the composition for improving the psychological state, the choline ester content is preferably 500 μg or more and 20 mg or less, and more preferably 1 μg or more and 10 mg or less. By orally ingesting at such a dose, a psychological state improving effect can be obtained more effectively.

In the present embodiment, the composition for improving a psychological state may be, for example, a dry powder such as a freeze-dried powder or a hot air-dried powder.

The composition for improving a psychological state of the present embodiment can be used as a food composition such as various functional health foods or pharmaceutical compositions.

In the case of food, it may be used in combination with a suitable food additive. Not limited to such food compositions, it can be blended into green tea, black tea, Oolong tea, miscellaneous grain tea, health drinks, sports drinks, and the like as a beverage, or blended into biscuits, bread, candy, chocolate, macaroons, and the like as a food. As a result. it is possible to provide it as a food in a form that can be ingested on a daily basis. It can also be used as a so-called supplement as an appropriate dosage form according to the preparation of the following pharmaceutical products.

In the case of a pharmaceutical product, it can be used in various dosage forms according to a usual dispensing method in combination with an appropriate pharmaceutical additive. Examples of such dosage forms include solid preparations such as powders, granules, capsules, pills, tablets, and the like, and oral administrations such as liquids, suspensions, emulsions, and the like.

When the composition for improving a psychological state according to the present embodiment is used as a food, it is used not only as a general food or drink, but also as a “Food for Specified Health Uses” or a “Food with Function Claims” that exerts a specific function to promote health.

In this case, specific embodiments include supplements consisting of capsules, tablets, powders, granules and the like containing the composition for improving a psychological state according to the present embodiment as an active ingredient: bakery foods such as bread, cakes, cookies and macaroons; seasonings such as sauces, soups, dressings, mayonnaise, and the like; dairy products such as milk, yogurt, creams and the like; confectionery such as chocolates, candy, and the like; or beverages such as green tea, black tea, Oolong tea, wheat tea, miscellaneous grain tea, fruit juice, vegetable beverages, dairy beverages, soft beverages, carbonated beverages and the like.

When the composition for improving a psychological state according to the present embodiment is used as an active ingredient of a pharmaceutical composition, the dose varies depending on the ratio of each component, and various factors such as the age, weight, sex, symptoms, administration method, and the like of a patient. The dose can also be increased or decreased as appropriate depending on the degree of symptom improvement. The number of administrations can be divided into 1 to several times a day.

When the composition for improving a psychological state according to the present embodiment is used as a food, the intake amount can be selected according to the case of oral administration of the above-mentioned pharmaceutical products. However, in the case of food and drink, unlike pharmaceutical products, the dose and frequency of administration are not particularly limited. Therefore, as long as no particularly serious symptom occurs, the intake amount may be selected without limitation in consideration of the purpose of maintaining health, as well as taste and preference.

The composition for improving a psychological state according to the present embodiment is used not only for the composition for improving a psychological state, but also for a subject with hypertension and a subject who is healthy (not hypertensive) but has high BP.

Various additives can be used and are not particularly limited, and for example, one or a mixture of two or more kinds of saccharides such as lactose hydrate, sucrose, glucose, reduced malt sugar, mannitol and sorbitol, and the like; starches such as corn starch, potato starch, partially pregelatinized starch, dextrin, pullulan, and the like and their derivatives; celluloses such as crystalline cellulose and microcrystalline cellulose; derivatives such as carboxymethyl cellulose; macrogol; and magnesium aluminometasilicate. Of these, dextrin is more preferred. One type of additive may be used alone, or two or more types may be used in combination. These additives are preferably contained in an amount of 1 to 1000 parts by mass, more preferably 10 to 300 parts by mass, per 100 parts by mass of the composition of the present invention.

The choline ester, which is the active ingredient of the composition for improving a psychological state according to the present embodiment, is not limited to the above-mentioned eggplant. Any of those derived from animals, those derived from plants, and those derived from microorganisms could be used. In particular, it is preferable to use one derived from an organism that humans have eating experience, and it is preferable to use one derived from an edible plant. That is, the composition for improving a psychological state according to the present embodiment is not limited to the fruit derived from eggplant, and may contain a water-soluble component of a food made from an agricultural product containing a high amount of choline ester as an active ingredient.

The edible plant other than eggplant is not particularly limited as long as it contains choline ester. Edible plants include, for example, cucumber, tomato, paprika, green pepper, asparagus, Japanese mountain yam, cabbage, lettuce, carrot, apple, shishito pepper, Japanese pear, grape, radish sprouts, broccoli, alfalfa, bean sprouts, buckwheat, bamboo shoots, and the like. Among them, it is preferable that the agricultural product contains a high amount of choline ester, and from the viewpoint of the content of acetylcholine, bamboo shoots and bamboo subfamily of Piceaceace (Poaceae, Bambusoideae, Bambuseae) are preferable, and bamboo shoots are preferably tips.

### 2. Method for Manufacturing a Composition for Improving a Psychological State

The psychological state improving composition according to the present embodiment is obtained in one embodiment by the following production method.

The method for producing the composition for improving a psychological state according to the present embodiment is to squeeze the eggplant fruit to obtain a juice, or to add water to the eggplant fruit, and to squeeze the water-added fruit. Even when it is produced from a raw material other than eggplant fruit, a composition for improving a psychological state can be obtained by a similar method.

The water to be added to the eggplant fruit is not particularly limited, and water in a temperature range of 5° C. or higher and 40° C. or lower may be added. It is preferable to add water at room temperature. The amount of water added is preferably 50 parts by mass or more and 200 parts by mass or less, more preferably 75 parts by mass or more and 150 parts by mass or less, and particularly preferably 100 parts by mass per 100 parts by mass of the raw eggplant fruit. If the amount of water is small, the extraction of the water-soluble component, which is an active ingredient, may be insufficient, and if the amount of water is large, the processing volume becomes large, and it may not be easy to process into a dry powder, for example.

The eggplant fruit is preferably crushed before or after the addition of water, and is particularly preferably crushed before the addition of water from the viewpoint of ease of crushing. Crushing the eggplant fruit facilitates the extraction of water-soluble components. The crushing can be performed in various forms such as strip cutting dice-shaped blocks, and paste-like forms, but it is preferable to crush them into paste-like forms in order to facilitate squeezing. For crushing, for example, a juicer, a mixer, a mill, a crusher or the like can be used.

Water used in the method for manufacturing the composition for improving a psychological state according to the present embodiment could be mineral water, distilled water, deionized water, ion-exchanged water, electrolyzed water, tap water, well water, industrial water used for food, and the like. Water generally used for food processing can be used. pH is not particularly limited, butin order to stably maintain ACh, the pH is preferably 9.0 or less and pH 3.0 or more, preferably pH 8.0 or less and pH 4.0 or more, and more preferably pH 6.5 or less and pH 4.5 or more. Water whose pH has been adjusted using a pH adjuster such as citric acid or ascorbic acid might be used.

In addition, ethanol may be added to reduce bacterial contamination during the juice squeezing operation. Increasing the ethanol concentration helps reduce microbial contamination, but the solubility of the water extract deteriorates and the extraction efficiency of water-soluble components decreases. Therefore, the ethanol concentration should be 9.9% or less, preferably 6% or less, further preferably 4% or less.

In the method for producing the composition for improving a psychological state according to the present embodiment, the eggplant fruit may be heated. By heating, the Ach degrading enzyme can be inactivated, and ACh can be efficiently extracted. In addition, the extraction of water-soluble components of the eggplant fruit and the crushing of the eggplant fruit can be facilitated as the eggplant fruit tissue is softened and the cell wall is destroyed.

The mode of heating is not particularly limited, and for example, heating can be performed for 2 to 60 minutes in a temperature range of 30° C. or higher and 120° C. or lower. The heating temperature is preferably 80° C. or higher and 100° C. or lower.

Examples of the method for heating eggplant fruits include a method of heating eggplant fruits in a microwave oven, a hot plate or a pot, a method of immersing eggplant fruits in boiling water, a method of applying steam, and the like. Among these, the method of immersing eggplant fruits in boiling water and the method of applying steam to the eggplant are preferable.

The eggplant fruit may be heated before, after the addition of water, during heating with water, before crushing after crushing or during crushing. It is preferable to crush the heated eggplant or to heat the crushed eggplant rapidly to inactivate the ACh degrading enzyme.

The method for squeezing the crushed eggplant fruit is not particularly limited as long as it can be separated into a juice and a residue. For example, the juice can be squeezed by a juice squeezing machine such as using a soup stock bag for wine, or by a method of squeezing juice with a fruit juice squeezer. A centrifuge or a screw press may be used.

The obtained juice can be dried into an aggregated or powdery dried product. The drying method is not particularly limited, and it is preferable that the moisture contained in the dried product can be dried to 0% by mass or more and 100% by mass or less. For example, it can be dried by drying by heating such as hot air drying other drying such as drum drying, freeze drying spray drying and the like.

The dried product can be crushed/ground into a dry powder. The crushing method is not particularly limited, but it is preferable that it can be crushed to about 5 to 100 mesh. For example, it can be pulverized using a mill.

The composition produced by the method for producing the composition for improving a psychological state according to the present embodiment tends to have a small amount of dietary fiber and a large amount of sugar. The ratio of dietary fiber to sugar in the composition of the present invention could be, for example, 1:2 to 1:100, preferably 1:5 to 1:40, and particularly preferably 1:10 to 1:20.

The composition for improving a psychological state containing eggplant-derived choline ester according to the present embodiment significantly improves the blood pressure and psychological state of a person with normal-high BP and a person with grade 1 hypertension who are feeling stressed by continuously ingesting the composition. This is demonstrated, for example, in a randomized, double-blind, placebo-controlled, parallel group comparison study as described in examples below.

For example, for the improvement of BP, when the composition for improving a psychological state according to the present embodiment is continuously ingested, the hospital BP (diastolic/diastolic after loading) and home BP (systolic/diastolic/diastolic at bedtime) is significantly improved for the participant with normal-high BP, and hospital BP (systolic/diastolic) is significantly improved for persons with grade 1 hypertension. As a whole, hospital BP (systolic/diastolic) and home BP (systolic/diastolic at wake up) are significantly improved.

Regarding the improvement of a psychological state, by continuously ingesting the composition for improving a psychological state according to the present embodiment, “depression-dejection”, “confusion-bewilderment”, “anger-hostility”, negative psychological states such as “TMD score” are significantly improved for a person with normal-high BP, and positive psychological states such as “friendliness” and “vigor-activity” are significantly improved for persons with grade 1 hypertension. Overall, “confusion-bewilderment” is significantly improved.

As will be described later, there are no issues in safety even if the composition for improving a psychological state according to the present embodiment is continuously ingested, and no side effects or problematic adverse events are observed during the ingestion period of 12 weeks.

As described above, the psychological state improving composition and the method for producing the psychological state improving composition according to the present embodiment could provide a new composition having a psychological state improving effect.

Since the psychological state improving composition according to the present embodiment is for oral ingestion, the effect of improving the psychological state can be obtained by eating the eggplant fruit itself as well. In this case, as described above, for example, by eating the eggplant fruit at a dose capable of ingesting a choline ester content of 5 g or more and 50 mg or less, the psychological state improving effect and other effects such as an effect of improving blood pressure can be obtained. Eggplants to be eaten can be effective even if they are eaten raw or heated, and even processed foods such as pickles can be effective.

### 3. Examples

Hereinafter, the present invention will be described in more detail with reference to examples and comparative examples. However, the present invention is not limited to these examples. In addition, “part” and “%” in examples and comparative examples are on a mass basis unless otherwise specified.

In this example, a randomized, double-blind, placebo-controlled, parallel-group comparative study is designed to investigate the long-term effect of eggplant of improving blood pressure, stress, and a psychological state of a human subject with stressed, normal-high BP and/or grade 1 hypertension.

BP is classified according to the Hypertension Treatment Guideline 2014 (edited by the Japanese Society of Hypertension's Hypertension Treatment Guideline Development Committee, April 2014, published by the Japanese Society of Hypertension, ISBN: 9784-8975-322-5) and is defined as follows.

Normal-high BP: 130 mmHg<systolic blood pressure (SBP)≤139 mmHg and/or 85 mmHg<diastolic blood pressure (DBP)≤89 mmHg
Grade 1 hypertension: 140 mmHg<SBP≤<159 mmHg and/or 90 mmHg<DBP≤99 mmHg

### 3.1 Materials and Methods

### 3.1.1 Study Population

We screened 189 volunteers on screening visit, and 100 Japanese participants (men and women between the ages of 35 and 65 who were stressed by normal-high BP or grade 1 hypertension) were enrolled in the study. Participants with any of the following were excluded from the study:
Participants under a physician's advice, treatment, or medication for hypertension, schizophrenia, depressive disorder, mania neurologic disorder, arrhythmia, or bradycardia.
Participants with a body mass index (BMI) of ≥30 kg/m², pre- or post-menopausal women complaining of obvious physical changes.
Participants at risk of having allergic reactions to drugs or foods, especially those based on eggplant, secondary hypertension, serious cerebrovascular, cardiac, hepatic, renal, gastrointestinal diseases.
Participants affected with infectious diseases requiring reports to the authorities.
Participants with history of major surgery relevant to the digestive system.
Participants with unusually high or low blood pressure, or abnormal hematological data.
Participants with severe anemia.
Participants who regularly received medicine, functional foods, or supplements that would affect BP or stress.
Participants with alcohol addiction or an eating disorder.
Participants who donated either 400 mL of whole blood within 16 weeks (women) or 12 weeks (men), 200 mL of whole blood within 4 weeks (men and women).
Participants who were participating in other clinical studies, or participated within the last 4 weeks prior to the current study.
Pregnant or lactating women.
Women who expected to be pregnant during this study.
Participants with any other medical or health reasons unfavorable in the current study.

Eligible participants were randomly assigned to either the eggplant (capsules containing eggplant powder) group or placebo (capsules containing dextrin powder) group, stratified by sex, age, and hospital SBP and DBP during the first visit. The characteristics of participants in each group are summarized in Table 1. Assignment of food was conducted through the use of block randomization method by using a computer at a third-party data center (Media Educational Center, Hokkaido Institute of Information Technology, Ebetsu, Hokkaido, Japan). Clinical research collaborators were blinded to the assignment information during the study period. The assignment information was opened after analysis data was finalized.

TABLE 1

| Characteristic | Unit | Placebo | Eggplant | p |
|---|---|---|---|---|
| Subjects | n | 41 | 36 | — |
| Male | n | 16 | 9 | 0.228 |
| Age | years | 54.4 ± 6.2 | 54.2 ± 7.2 | 0.886 |
| Height | cm | 163.1 ± 8.4 | 159.6 ± 7.5 | 0.065 |
| Body mass index | kg/m² | 22.9 ± 2.7 | 22.5 ± 3.2 | 0.596 |
| Visual analogue scale | mm | 42.4 ± 18.5 | 41.9 ± 16.3 | 0.901 |
| Hospital systolic | mmHg | 138.3 ± 8.8 | 137.1 ± 7.8 | 0.518 |
| blood pressure | | | | |
| Hospital diastolic | mmHg | 86.1 ± 8.5 | 85.0 ± 7.9 | 0.578 |
| blood pressure | | | | |
| Intake rate | % | 99.0 ± 1.8 | 98.9 ± 1.8 | 0.947 |

### 3.1.2. Power Analysis of Sample Size

We calculated that a sample size of 80 (40 in each group) was necessary using the effect size of 0.63 which was detected by the result of pilot study over an 8 week treatment period (registered at https://upload.umin.ac.jp/cgi-open-bin/ctr_e/ctr_view.cgi?recptno=R000032943; registration number, UMIN000028785; date of registration, Aug. 13, 2017) with a statistical power of 80% and an α of 5%. Assuming a 20% loss in the follow-up rate, 100 participants (50 in each group) were enrolled.

### 3.1.3 Preparation of Eggplant Powder and Capsules

The composition for the study was prepared by the following method.

Eggplant powder was made from eggplant fruit juice containing excipient through dry powdering. The test food was defined as a daily dose of eggplant-derived choline ester (ACh). The amount of ACh was set as 2.3 mg/day. (which corresponds to approximately 22 g of the fresh eggplant used in the current study.)

Eggplants cultivated in Kochi prefecture (Japan) were used as raw materials. Fresh eggplant fruits were washed, cut into pieces, heated to prevent degeneration, and then squeezed to remove the pulp, generating eggplant juice. Dextrin was added to the juice, which was then lyophilized. This lot of lyophilized eggplant powder containing 1.92 mg/g of eggplant-derived choline ester (ACh) was obtained with a yield of 4.08% from the raw materials, and then encapsulated to afford 300 mg capsules. Placebo capsules contained only dextrin powder, without eggplant powder.

The eggplant and placebo capsules were prepared under strict quality-controlled protocols and were identical in appearance. Analyses of the nutrient composition of eggplant and placebo capsules were conducted using the methods established by the Japan Food Research Laboratories (Tokyo, Japan), and the results are presented in Table 2. ACh was measured by using liquid chromatography-tandem mass spectrometry (LC-MS/MS) at Shinshu University. In the clinical trial, the participants received daily ingestion of 1200 mg eggplant powder (300 mg powder containing 25% dextrin×4), which was produced from approximately 22 g of the fresh eggplant.

TABLE 2

| Composition | Placebo | Eggplant |
|---|---|---|
| Calories (kcal) | 4.35 | 4.39 |
| Water (g) | 0.03 | 0.03 |
| Proteins (g) | 0.00 | 0.11 |
| Lipids (g) | 0.00 | 0.02 |
| Carbohydrates (g) | 1.09 | 0.95 |
| Ash (g) | 0.00 | 0.09 |
| Sodium (mg) | 0.00 | 0.30 |
| Choline ester (Acetylcholine, mg) | 0.00 | 2.30 |
| γ-Aminobutyric acid (GABA, mg) | 0.00 | 7.65 |
| Chlorogenic acid (CA, mg) | 0.00 | 12.3 |

### 3.1.4. Treatment

This clinical trial was conducted at Hokkaido Information University, Health Information Science Research Center. The participants ingested four eggplant powder capsules containing 2.3 mg of ACh or four placebo capsules every day for 12 weeks. Instructions were to swallow two capsules in the morning (after breakfast) and two capsules in the evening (before sleep). Medical interviews, vital sign and body composition measurements, urinary assessments, hematological and biological assessments, and questionnaire completion were conducted during the baseline, week 4, week 8, week 12, and week 16 (four weeks after the end of ingestion). During the entire course of this study, participants were asked to maintain their daily activities, including food consumption and exercise habits, and to avoid consuming any supplements, eggplant, and bamboo shoot, including processed foods containing them, and they recorded their daily activities. Bamboo shoot is known to contain a similar level of ACh as eggplant, and the test food was the only ACh-rich food allowed to be consumed by the participants.

The primary outcome assessed was hospital BP comprising SBP and DBP. The secondary outcomes assessed were home BP (morning and evening), stress assessed by using the visual analogue scale (VAS) questionnaire, and PSs assessed by using the full-length version of the ‘Profile of Mood States 2^{nd} Japanese Edition (POMS-2) for Adults’.

### 3.1.5. Quantitation of Hypotensive Ingredients in Eggplant Capsules

The quantitation of ACh was performed by the standard addition method according to the method described in our applied patent WO2018070545 with minor changes. A portion of the test food was added to (2-aminoethyl) trimethylammonium pivaloylamide (EN; internal standard, synthesized at Shinshu University), was mixed with 100 mM phosphate buffer, and applied to a weak acidic cation exchange cartridge (Inertsep CBA 100 mg/l mL; GL Science Inc., Tokyo, Japan). The fraction containing ACh was eluted with 1 M HCl and collected into a measuring flask. After being filled up to appropriate amount with the mobile phase, the solution was dispensed into three aliquots and spiked with different amount of standard ACh to make dilution series.

ACh and EN in the samples were analyzed by LC-MS/MS with following condition. The separation was performed using isocratic mobile phase (33% (v/v) methanol containing 0.010%0 formic acid) on a YMC Triart-PFP column (4.6 mm×250 mm, 5 μm; YMC. Co., Ltd., Kyoto, Japan). ACh and EN were detected by positive multiple reaction monitoring mode (MRM) at the following MRM transitions:

146.15>87.10 (ACh); and

187.30>128.15 (EN).

The quantitative value of ACh was corrected by the recovery of EN. The quantitation of γ-aminobutyric acid (GABA) and chlorogenic acid (CA) was also performed in a similar manner at the following MRM transitions:

104.15>87.20 (GABA); and

355.10>163.20 (CA)

### 3.1.6. Hospital BP and Home BP Assessments

Hospital BP was measured by a doctor or nurse using an Automatic Blood Pressure Monitor HEM-7080IC (Omron Healthcare Co., Ltd., Kyoto, Japan) using the upper arm of the nondominant arm after a >10-min rest. Three sequential measurements were performed, and the median of the measurements was taken at each evaluation point.

Home BP was measured by the participants using an Automatic Blood Pressure Monitor HEM-7080IC using the upper arm of the nondominant arm. Participants measured BP daily for one week prior to visits 2-6, within 1 h after waking up (morning BP), and before going to bed (night BP). Three sequential measurements were performed, and the median of the measurements was taken each day. The average BP during three days prior to each evaluation point was evaluated.

### 3.1.7. Physical, Hematological, Biochemical, and Urinary Tests

Blood was collected from the participants after a 12 h fasting and used for the following hematological examinations: white blood cell (WBC), red blood cell (RBC), hemoglobin (Hb), hematocrit (Hct), and blood platelet (Plt) count. Biological examinations included liver function (aspartate aminotransferase (AST), alanine aminotransferase (ALT), gamma-glutamyl transpeptidase (γ-GTP), alkaline phosphatase (ALP), and lactate dehydrogenase (LDH) levels); renal function (blood urea nitrogen (BUN), creatinine (CRE), and uric acid (UA) levels); lipid profiles (total cholesterol (TC), low-density lipoprotein cholesterol (LDL-C), high-density lipoprotein cholesterol (HDL-C), and triglyceride (TG) levels); and blood glucose profiles (fasting plasma glucose (FPG) and hemoglobin (Hb) Alc levels).

First void urine was collected, and the sample was used for qualitatively assessments of pH, sugar, protein, occult blood, urobilinogen, and ketones.

Blood and urine tests were analyzed at Sapporo Clinical Laboratory, Inc. (Hokkaido, Japan). Body composition (BW, body fat rate (BFR), and BMI) was assessed using a Body Composition Analyzer DC-320 (Tanita Corp, Tokyo, Japan).

### 3.1.8. VAS Questionnaire Assessing Stress and Profile of PSs by POMS-2

We used a VAS questionnaire to assess participants' stress. Participants were instructed to place an “X” along a 100 mm line to provide a rating from the worst condition (the left end, 0 mm) to best condition (the right end, 100 mm) for each question based on their current health condition. The questionnaire results were assessed by evaluating the length from the beginning of the line on the left to the “X”, and the increase of score considered as improvement stress.

The POMS-2 questionnaire was used to evaluate the effects of eggplant on PSs. Participants were instructed to complete it prior to taking the capsules. Anger-hostility, confusion-bewilderment, depression-dejection, fatigue-inertia, and tension-anxiety were defined as negative PSs, whereas vigor-activity and friendliness were defined as positive PSs.

All PSs were assessed using the POMS-2 full version for adults, which comprised 65 questions (Success Bell, Tokyo, Japan). Participants selected one of five answers ranging from “not at all” to “quite a lot” based on their PS over the previous week. Total mood disturbance scores, which were comprehensively expressed as negative PSs, were calculated by adding anger-hostility, confusion-bewilderment, depression-dejection, fatigue-inertia, and tension-anxiety scores, and then subtracting the vigor-activity and friendliness scores.

For assessing the antistress effects of eggplant, participants first ingested two capsules and then completed the Uchida-Kraepelin Psychodiagnostic Test (UKT) for 15 min to induce mental stress. The VAS questionnaire was completed two times: before intake of test food and after completion of the UKT.

### 3.1.9. FFQg

In addition, all participants completed a Food Frequency Questionnaire Based on Food Groups (FFQg) (Kenpakusha, Tokyo, Japan) during the second to sixth visits. The FFQg was used to estimate nutrient (calories, protein, fat, carbohydrates, dietary fiber, and salt) intake based on the participants' regular diet. Participants reported their weekly amount and frequency of food intake about 29 food groups and 10 types of cooking methods.

### 3.1.10. Ethics

The current clinical study was conducted in accordance with the Declaration of Helsinki and the ethical guidelines on medical research with humans (Ministry of Education, Culture, Sports, Science and Technology, and Ministry of Health, Labor and Welfare). Written informed consent was obtained from participants after reading a study consent form of clinical trial prior to being enrolled. The study protocol was approved by the ethics committee of Hokkaido Information University (Ebetsu, Hokkaido, Japan; approved on May 25, 2018; approval number: 2018-06). This study is registered at https://upload.umin.ac.jp/cgi-open-bin/ctr_e/ctr_view.cgi?recptno=R000037995 (registered on Jul. 10, 2018; registration number: UMIN000033330).

### 3.1.11. Statistical Analysis

Student's t-test was used to analyze the primary and secondary outcomes, safety outcomes, and food frequency questionnaire values by comparing the changes in participant values between the two groups. Changes in participant values were analyzed using repeated measures of analysis of variance between groups. For participant characteristics, Fisher's exact probability test was used for sex and the Mann-Whitney U test was used for intake rate; Student's t-test was used for other participant characteristics. In a subgroup analysis, we analyzed hospital BP in participants with SBP of 130-139 mmHg or DBP of 85-89 mmHg (normal-high BP participants) and those with SBP of 140-159 mmHg or DBP of 90-99 mmHg (grade 1 hypertension participants). All statistical analyses were performed using SPSS v25 (IBM Japan, Ltd., Tokyo, Japan). Exploratory data analysis using measured values were also performed in the same manner. All results are expressed as the mean±standard error. It was considered a statistically significant difference when p-value was less than 0.05.

### 3.2. Results

### 3.2.1. Participant Dropouts and Characteristics

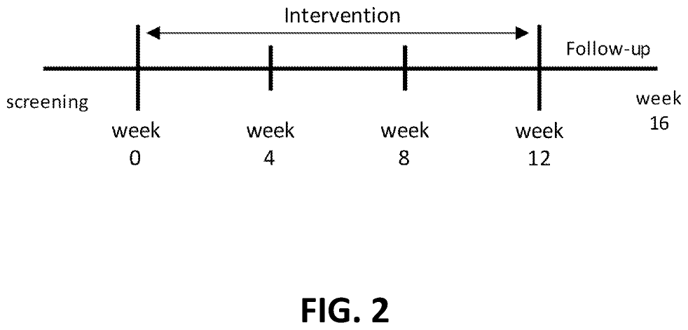

Participant involvement throughout the study period and study schedule are shown in FIG. 2. Volunteers who provided informed consent (n=189) were assessed for eligibility, and after screening, 100 participants were enrolled in this study. All enrolled participants were randomized into one of two intervention groups (placebo group, n=50; eggplant group, n=50). Four participants (personal reason, n=4) dropped out prior to study initiation, and nine participants dropped out during the study for the adverse events or personal reason (placebo group, adverse event such as pneumonia (n=1), colonic diverticulitis (n=1), dyslipidemia (n=1), and hives (n=1); eggplant group, adverse event such as injury (n=2), mild liver dysfunction (n=1) and nephropathy (n=1), personal reason (n=1)). Finally, 87 participants completed this study: 42 in the eggplant group and 45 in the placebo group. Ninety-six participants, excluding the four participants who dropped out because of personal reasons before the start of the study, were included in the safety analysis. We excluded 10 participants in the efficacy analysis because of abnormal variation (outside of the reference range (n=1), and >120%0 changes compared to the baseline among safety outcomes (n=1)) in values or mild sickness (allergies unrelated to the test food (n=2)), compliance problems, or participant not meeting the criteria (n=3), without stress at the beginning of treatment (n=1), missing primary outcomes owing to the absence of BP measurement (n=2). The efficacy analysis comprised of 36 participants in the eggplant group and 41 in the placebo group. Sex ratio, mean age, height, BMI, hospital SBP, hospital DBP, and intake rate for each group are shown in Table 1. There were no differences between the two groups regarding these characteristics.

### 3.2.2. Efficacy of Eggplant on Hospital BP

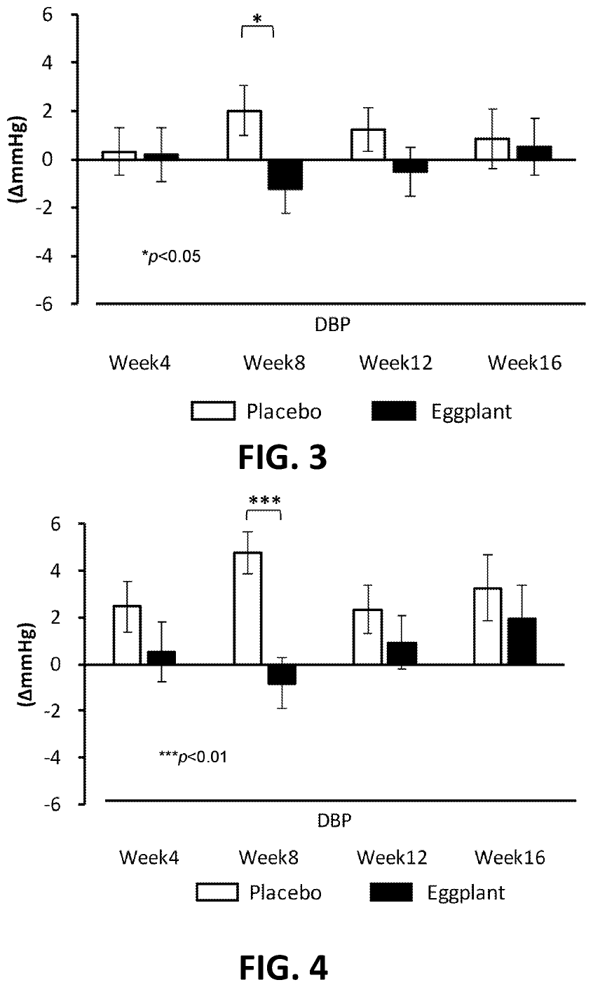

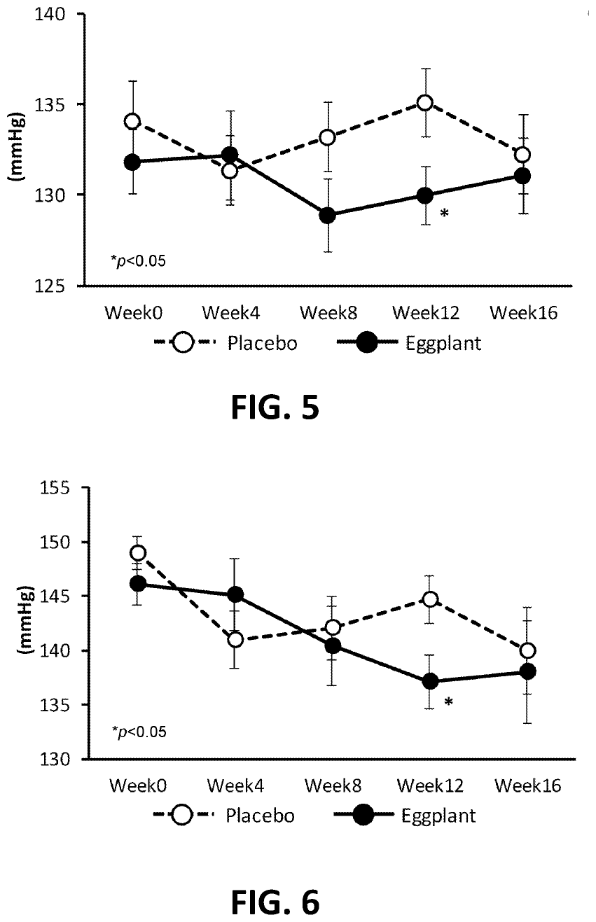

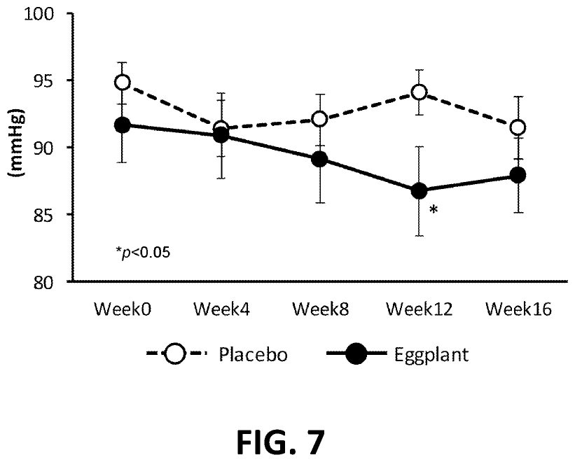

FIGS. 3 to 7 show the amount of change in Hospital BP from week 0 (FIGS. 3 and 4) and the measured value (FIGS. 5 to 7). FIG. 3 shows DBP of all subjects, FIG. 4 shows DBP of normal-high BP participants, FIG. 5 shows DBP of all participants, FIG. 6 shows SBP of grade 1 hypertensive participants, and FIG. 7 shows DBP in grade-1 hypertensive participants. Values are displayed as mean±standard error. Statistical significance by Student's t-test: * p<0.05 and *** p<0.001 vs placebo group.

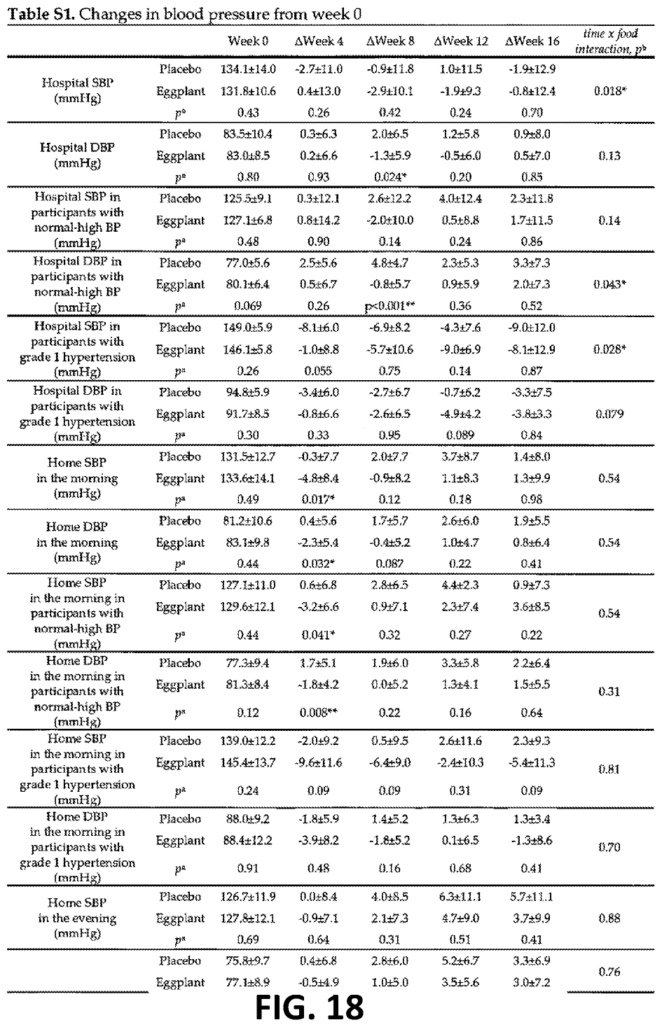

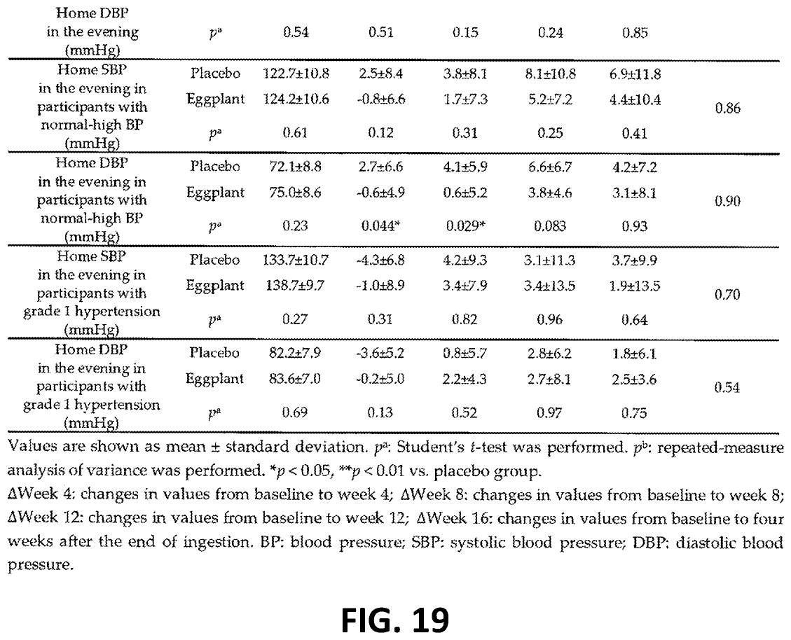

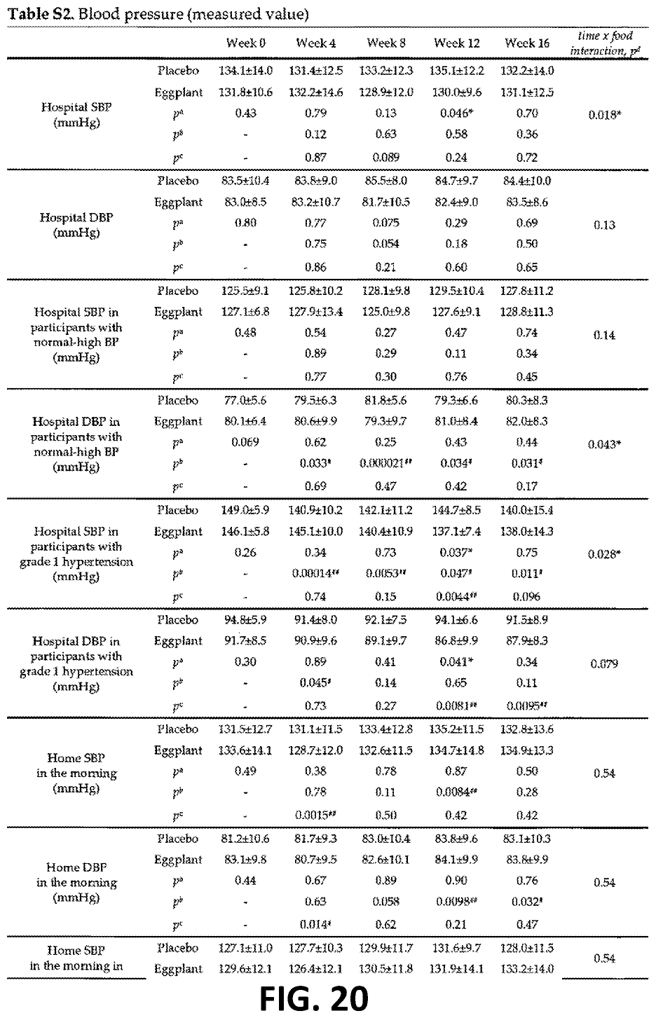

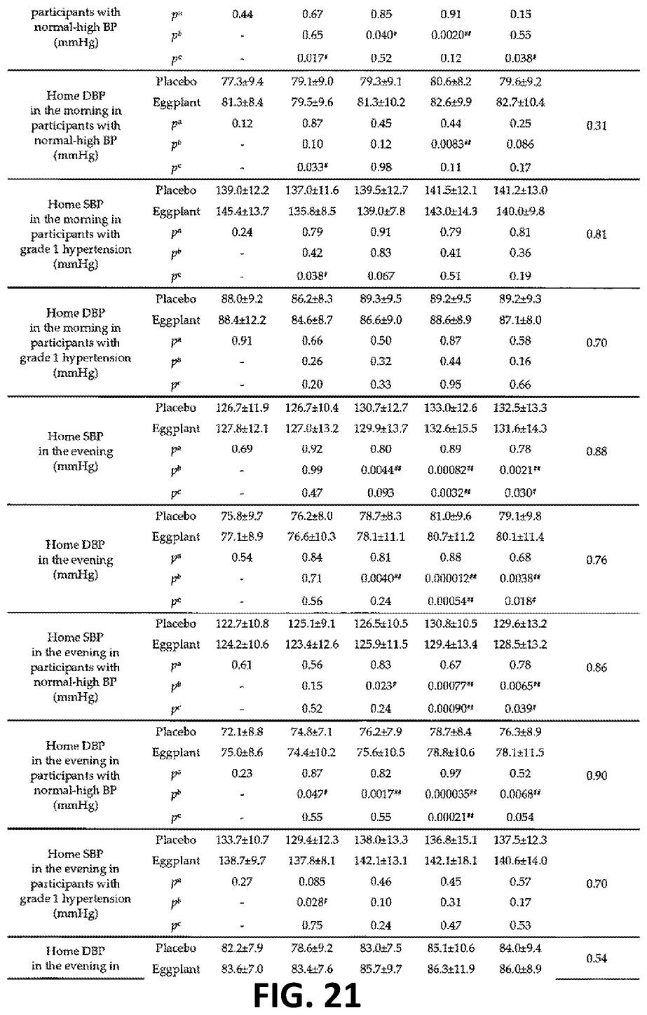

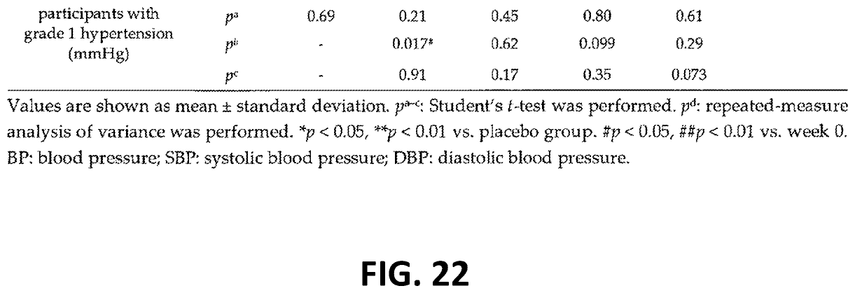

Eggplant powder intake significantly improved DBP at week 8, compared to that in the placebo group (p=0.024) (FIG. 3, Table S1; FIGS. 18, 19). In a subgroup analysis, the DBP increase was significantly suppressed at week 8 (p<0.001) following ingestion of eggplant in participants with normal-high BP (placebo: n=26; eggplant: n=27) (FIG. 4, Table S1; FIGS. 18, 19). Exploratory data analysis showed significantly lower SBP at week 12 in the eggplant group than in the placebo group (p=0.046)(FIG. 5, Table S1; FIGS. 20-22). The SBP and DBP of participants with grade 1 hypertension (placebo: n=15; eggplant: n=9) were significantly lower at week 12 in the eggplant group than in the placebo group (SBP: p=0.037, DBP: p=0.041)(FIGS. 6, 7, Table S2; FIGS. 20-22). Moreover, the significance of the time-food interaction of hospital SBP overall (change and measured value, p=0.018) and the grade 1 hypertension group (change and measured value, p=0.043) and hospital DBP in the normal-high BP group (change and measured value, p=0.028) also supported the BP lowering effect of the eggplant powder ingestion (Table S1; FIGS. 18, 19 and Table S2; FIGS. 20-22).

### 3.2.3. Efficacy of Eggplant on Home BP

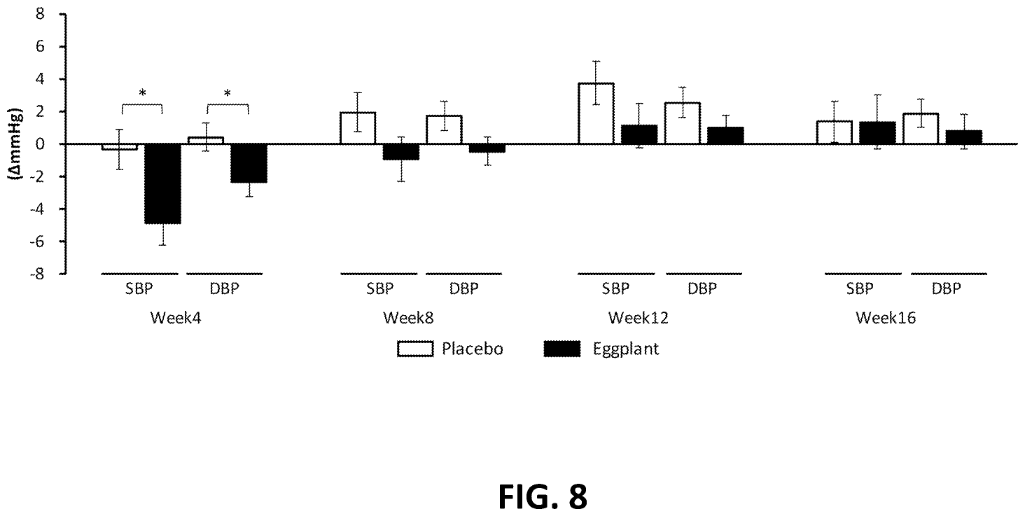

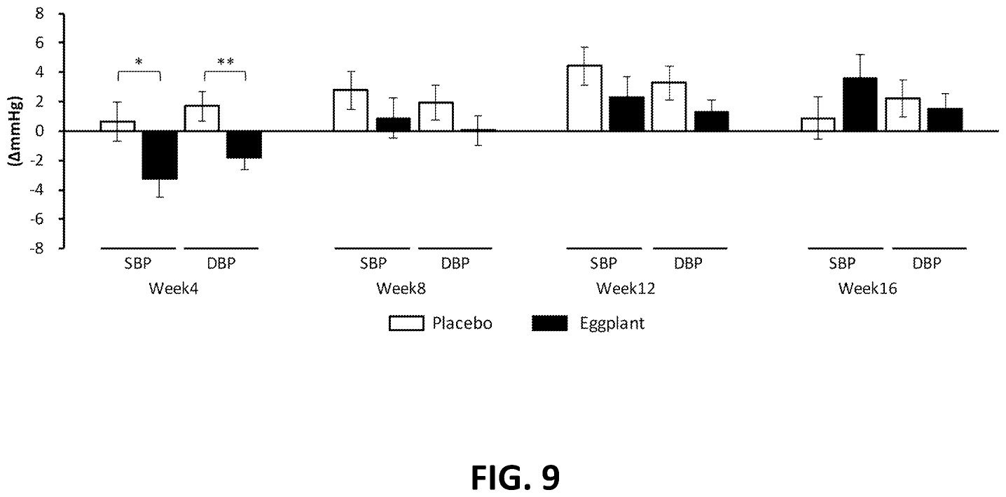

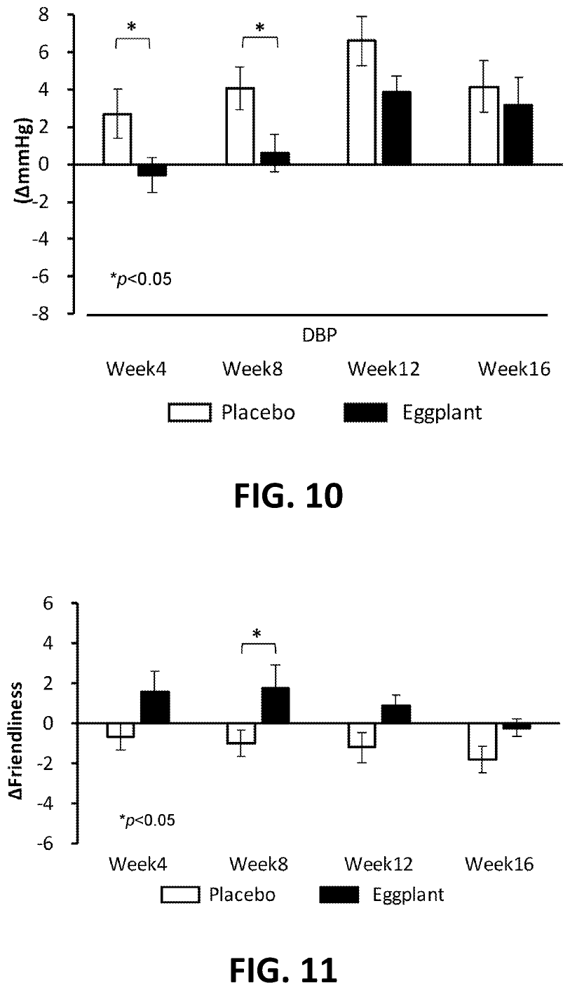

FIGS. 8 to 10 show the amount of change in home BP from week 0. FIG. 8 shows the SBD and DBP when all participants wake up, FIG. 9 shows the SBP and DBP when the participants wake up, and FIG. 10 shows the DBP at bedtime of the normal-high BP participants. Values are displayed as mean±standard error. Statistical significance by Student's t-test: * p<0.05 and *** p<0.01 vs placebo group.

Intake of eggplant powder significantly decreased morning SBP (FIG. 8 p=0.017) and DBP (FIG. 8, p=0.032) at week 4, compared to those in the placebo group. In addition, subgroup analyses showed a significant decrease in morning SBP (FIG. 9, p=0.041) and DBP (FIG. 9, p=0.008) at week 4 and significant suppression in the evening DBP increase at week 4 and week 8 (FIG. 10, p=0.029) in normal-high BP participants in the eggplant group compared to those in the placebo group. There was no significant difference in the measured values between the placebo and eggplant groups.

### 3.2.4. Efficacy of Eggplant on Stress and BP

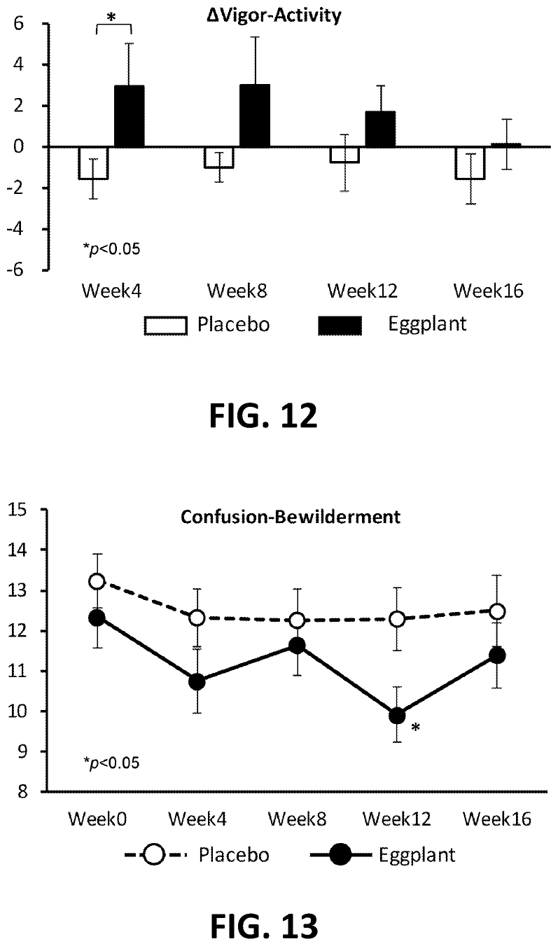

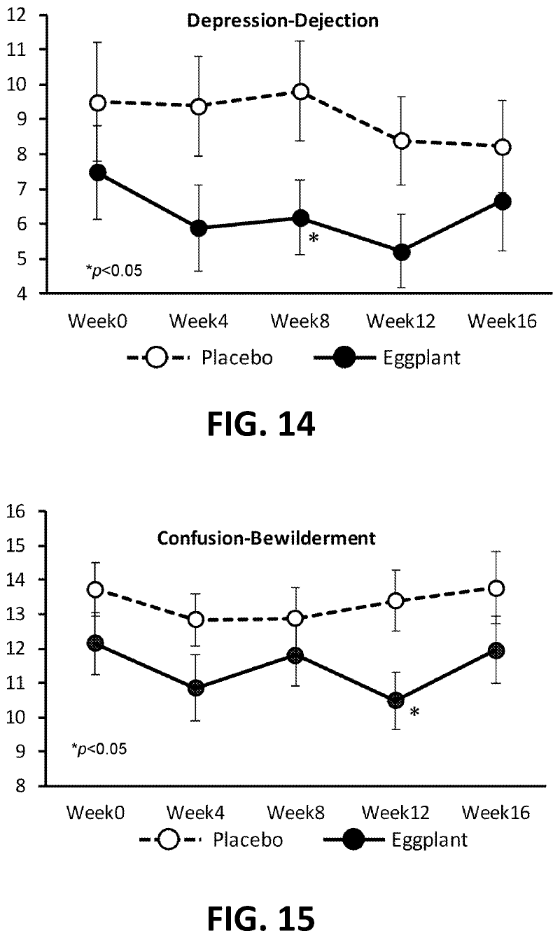

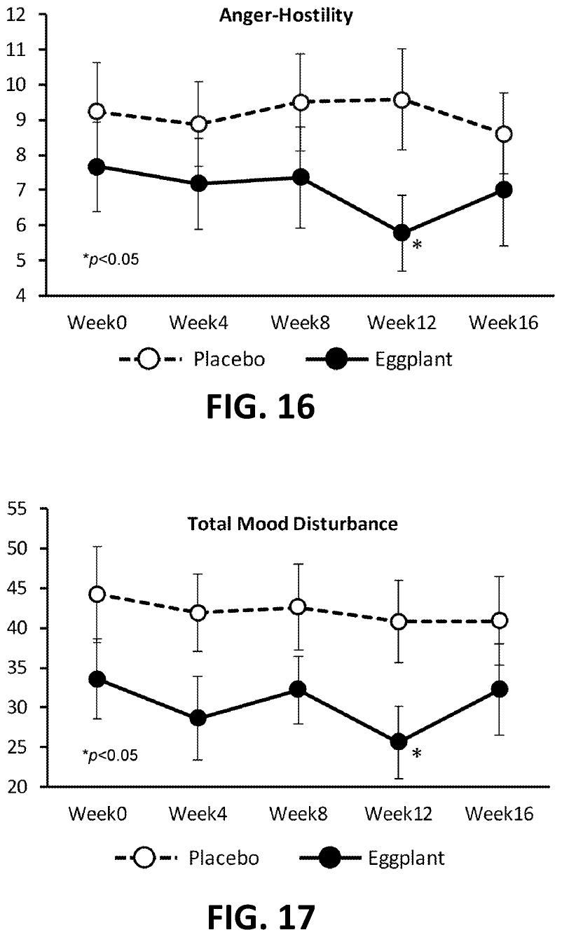

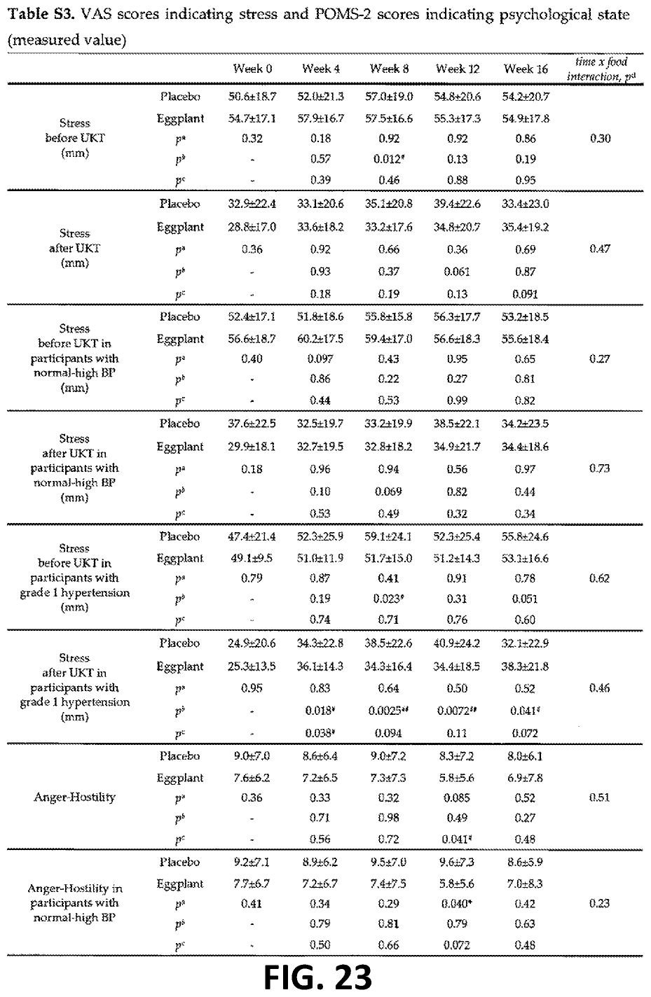

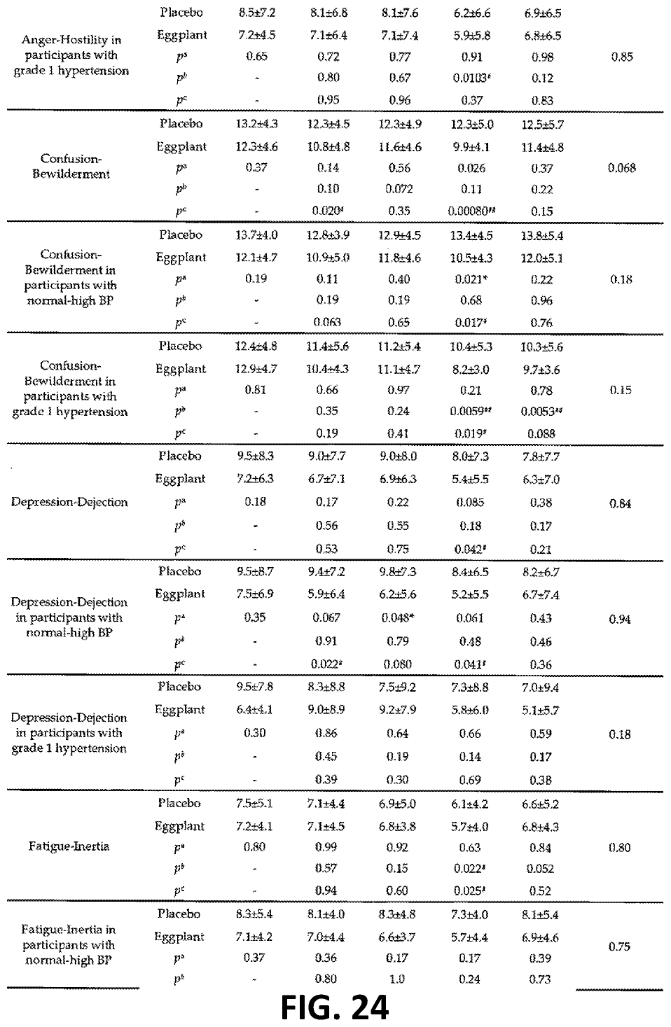

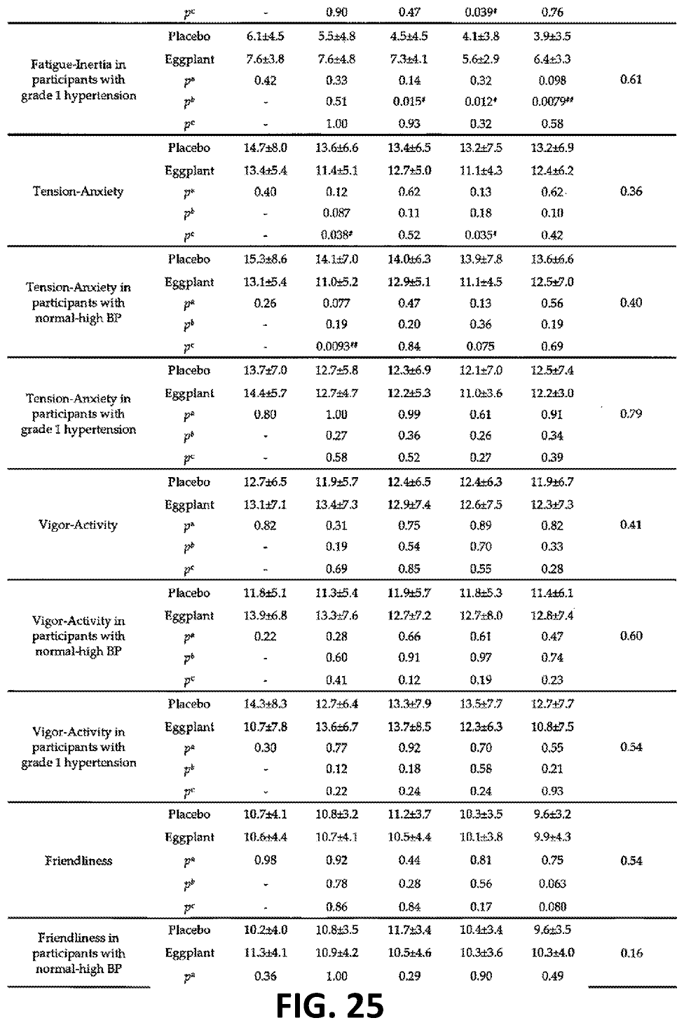

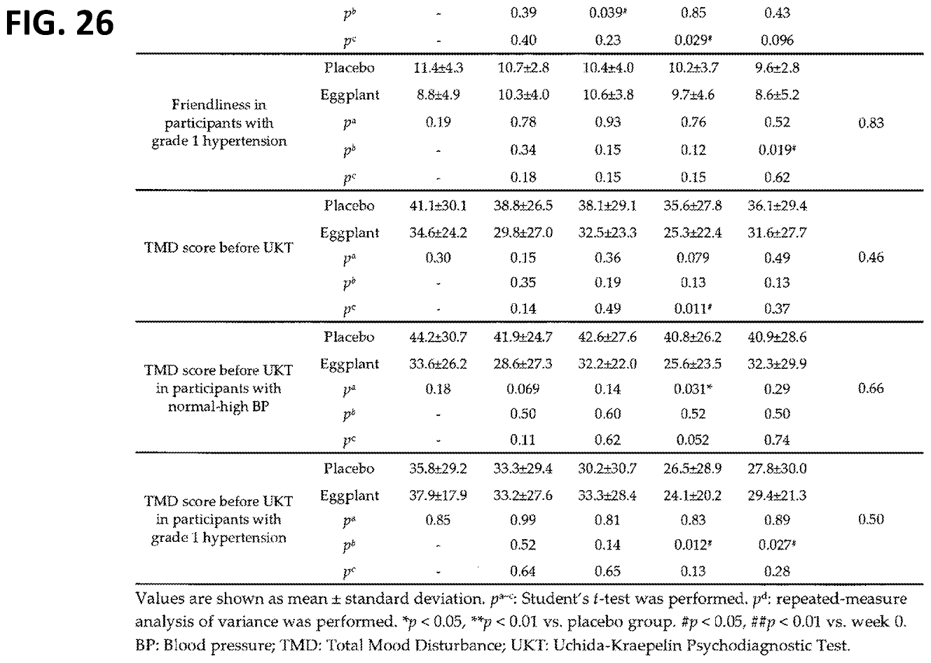

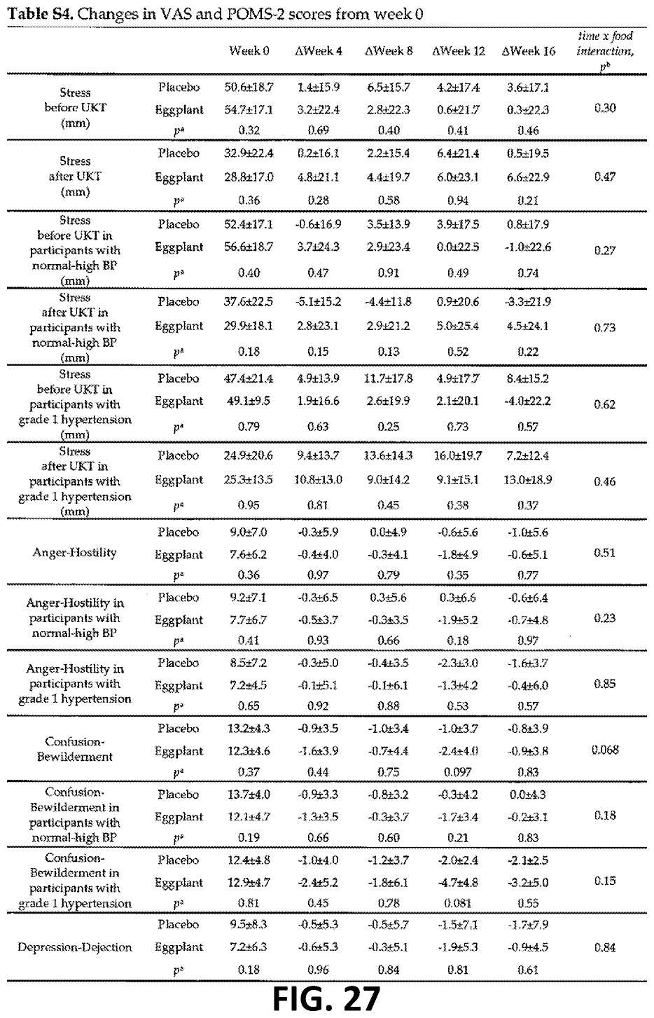

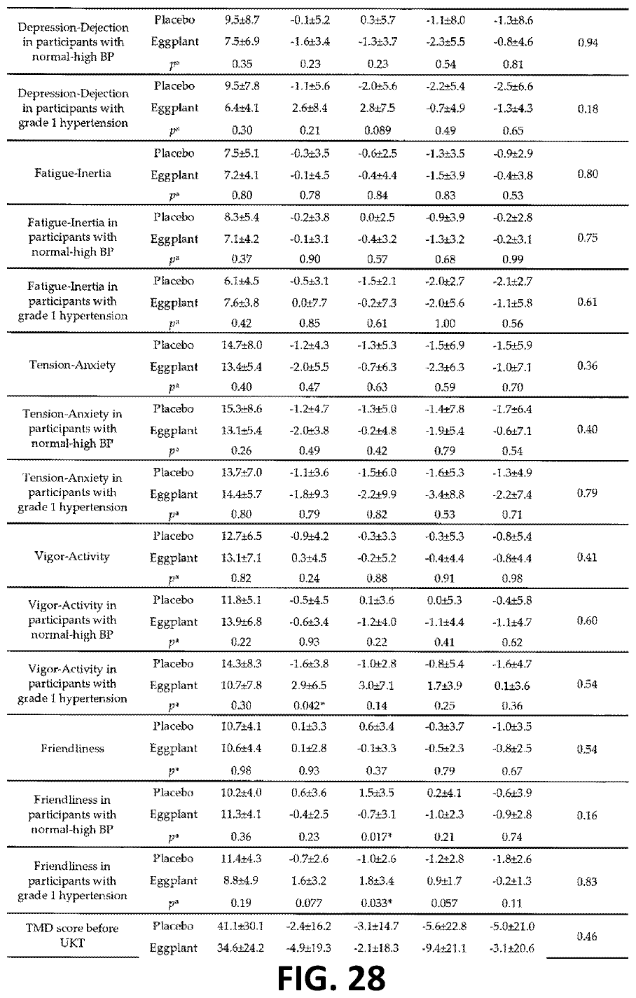

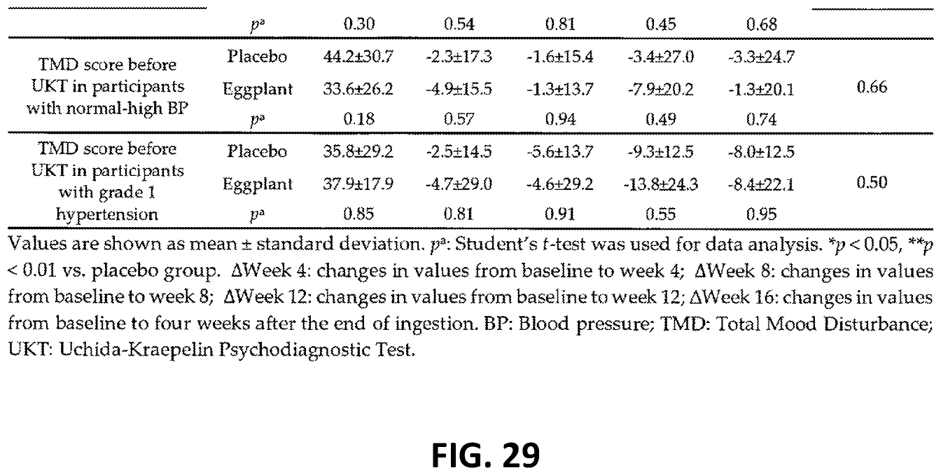

To assess the effects of eggplant on stress and PSs, changes in VAS scores, which indicated stress, and POMS-2 scores, which indicated PSs, were evaluated (Table S3; FIGS. 23-26, and S4; FIGS. 27-29). There were no differences in VAS scores between the eggplant and placebo groups, both before and after UKT. Subgroup analyses showed that the difference in POMS-2 scores from week 0, ‘vigor-activity’ at week 4, and ‘friendliness’ at week 8 of participants with grade 1 hypertension in the eggplant group were significantly superior to those in the placebo group (FIG. 4). Exploratory data analysis showed significant improvement in ‘confusion-bewilderment’ in all participants at week 12 and negative PSs (‘depression-dejection’ at week 8; ‘confusion-bewilderment’, ‘anger-hostility’, and ‘total mood disturbance’ at week 12) in participants with normal-high BP in the eggplant group (FIGS. 11-17).

### 3.2.5. Assessment of Dietary Nutrients of Participants During the Study

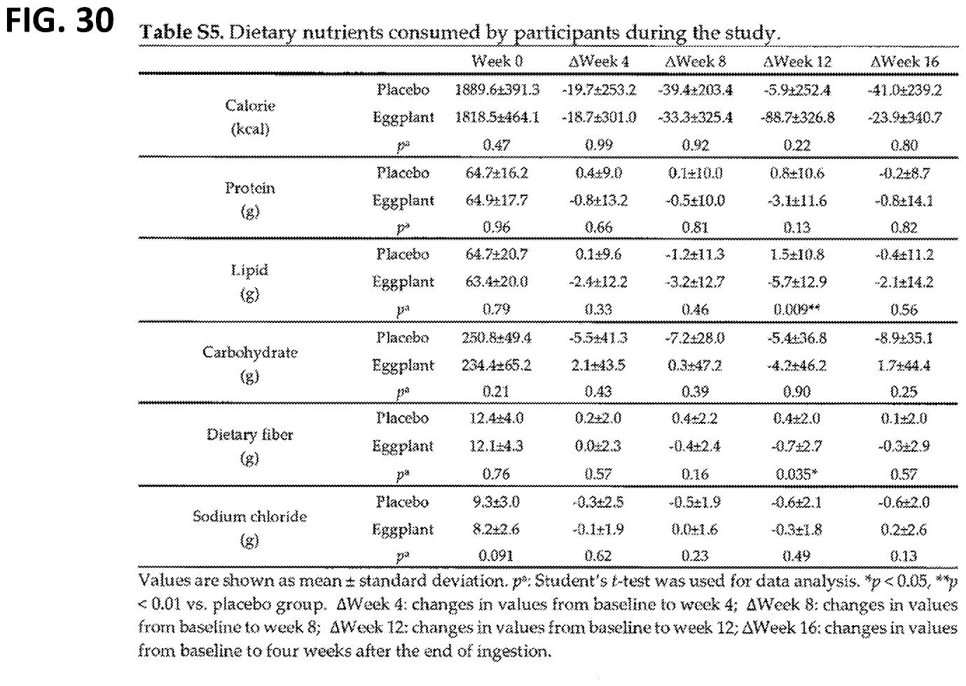

For the assessment of dietary nutrients, the participants completed FFQg (Table S5; FIG. 30). Lipid and dietary fibers at week 12 in the eggplant group were significantly lower than those in the placebo group (p=0.009 and 0.035, respectively). However, there were no statistically significant differences in the intake of calories, proteins, carbohydrates, and salt between the eggplant and placebo groups.

### 3.2.6. Assessment of Safety of Eggplant

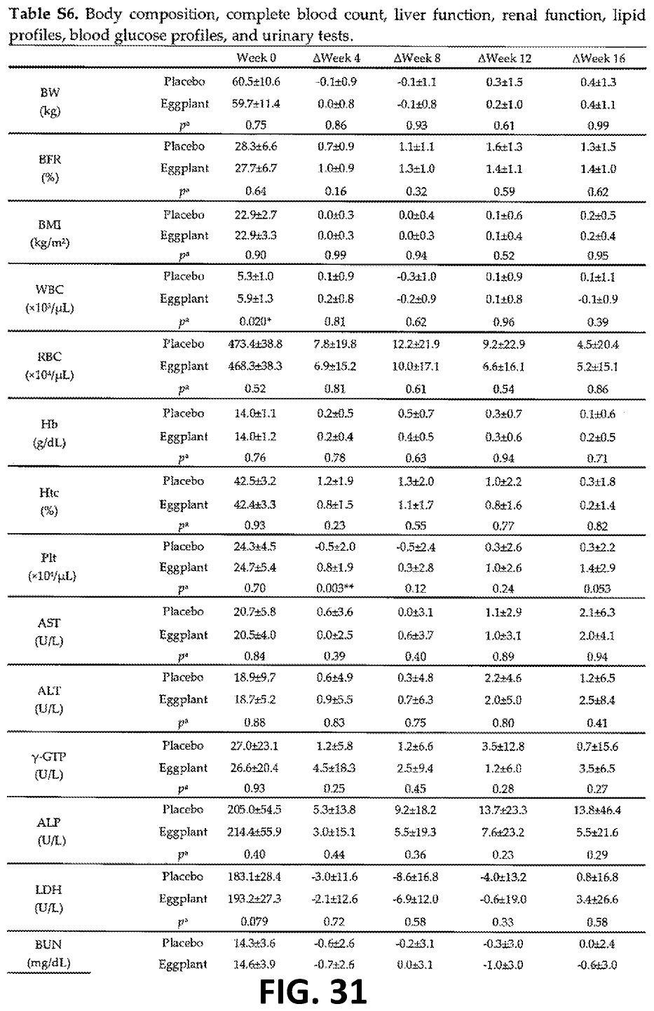

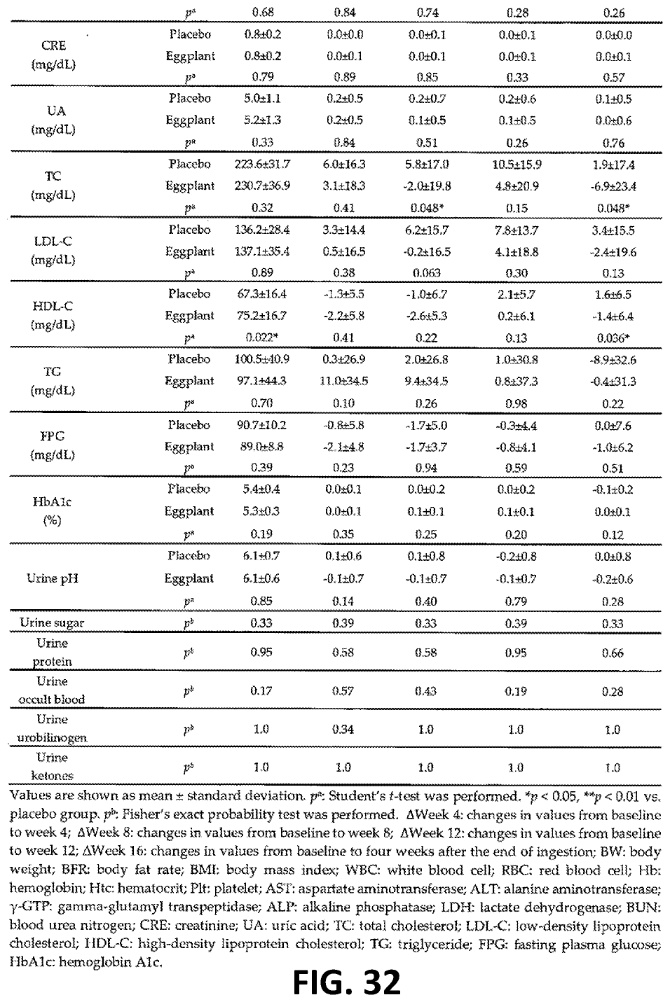

To analyze the safety of eggplant, we evaluated the body composition, complete blood count, liver function, renal function, lipid profiles, and blood glucose profiles of participants (Table S6; FIGS. 31, 32). Additionally, we qualitatively assessed urinary parameters (Table S6; FIGS. 31, 32). All changes in the values were within the reference value ranges. Adverse effects were observed in each group. However, the principal investigator inferred that all adverse effects were not related to the test food according to the protocol criteria. Thus, the eggplant powder intake had no or minimal unfavorable effects, at the raw eggplant dose of 22 g/day, which contained 2.3 mg of eggplant-derived choline ester (ACh).

### 3.3. Discussion

The randomized, double-blind, placebo-controlled, parallel group comparison study showed that continuous intake of eggplant powder containing 2.3 mg/day of eggplant-derived choline ester (ACh) improved BP in participants with normal-high BP and grade 1 hypertension. Subgroup analysis confirmed the effect of the eggplant powder on normal-high BP participants. A thorough search of the literature confirmed that this is the first clinical trial providing evidence of the beneficial effects of eggplant on BP. In addition, this study showed that eggplant intake improved positive and negative PSs.

Hospital DBP, which is the primary outcome in this study, was improved overall and specifically in the normal-high BP group. Interestingly, the improvement in DBP upon eggplant intake was more significant in the normal-high BP group (p<0.001) than overall (p=0.024). Improvements in hospital SBP overall and in the grade 1 hypertension group, as well as hospital DBP in the grade 1 hypertension group, were also shown through exploratory data analysis. Eggplant intake improved DBP in the normal-high BP group, and SBP and DBP in the grade 1 hypertension group, indicating that eggplant exerted different BP-improving effects for different BP categories.

The secondary outcomes, home SBP and DBP, were also improved by eggplant intake. The morning SBP and DBP overall and in the normal-high BP group, as well as the evening DBP of the normal-high BP group were also improved, supporting the hospital BP-improving effects of the eggplant intake.

Management of morning BP is important in heart failure prevention [White, W. B. Clinical assessment of early morning blood pressure in patients with hypertension. Prev. Cardiol. 2007, 10, 210-214., Wang, J. G.; Kario, K.; Chen, C. H.; Park J. B.; Hoshide, S.; Huo, Y.; Lee, H. Y.; Li, Y.; Mogi, M.; Munakata, M.; et al. Management of morning hypertension: A consensus statement of an Asian expert panel. J. Clin. Hypertens. 2018, 20, 39-44.]. It has also been reported that morning and night BPs of patients with anxiety disorder are significantly higher than those of healthy participants [Kayano, H.; Koba, S.; Matsui, T.; Fukuoka, H.; Toshida, T.; Sakai, T.; Akutsu, Y.; Tanno, K.; Geshi, E.; Kobayashi, Y. Anxiety disorder is associated with nocturnal and early morning hypertension with or without morning surge: Ambulatory blood pressure monitoring. Circ. J. 2012, 76, 1670-1677., Mellman, T. A.; Brown, D. D.; Jenifer, E. S.; Hipolito, M. M. S.; Randall, O. S. Posttraumatic stress disorder and nocturnal blood pressure dipping in young adult African-Americans. Psychosom. Med. 2019, 71, 627-630.], and one of the causes was estimated to be sleeping disorder due to sympathetic overactivity [Veith, R. C.; Lewis, N.; Linares, O. A.; Barnes, R. F.; Raskind, M. A.; Villacres, E. C.; Murburg, M. M.; Ashleigh, E. A.; Castillo, S.; Peskind, E. R.; et al. Sympathetic nervous system activity in major depression: Basal and desipramine-induced alterations in plasma norepinephrine kinetics. Arch. Gen. Psychiatry. 1994, 51, 411422.]. Thus, we speculated that eggplant intake suppressed sympathetic nervous activity in the participants during sleep, thereby improving the morning BPs of participants with stress.

Previously, we reported the BP-lowing effect of eggplant in SHRs [16]. Daily oral intake of low-dose eggplant powder (0.821 mg/kg BW) significantly prevented increases in both the SBP and DBP of SHRs, and the effective dose of the eggplant powder for an adult person (60 kg) was calculated to be 13.1 mg/day by Kleiber's law [Kleiber, M. The Fire of Life: An Introduction to Animal Energetics; Wiley: New York, N.Y., USA, 1961.]. The efficacy of ACh in eggplant may be lower in human participants with normal-high BP and grade 1 hypertension than in SHRs that developed high-grade hypertension. In the SHR study, urinary catecholamine level was decreased by ACh in eggplant by acting on the M3 muscarinic ACh receptor (M3 mAChR), suggesting that ACh in eggplant lowers BP by suppressing sympathetic nervous activity through M3 mAChR in the digestive system. M3 mAChR is present on epithelial cells of the stomach and intestine [Wess, J. Muscarinic acetylcholine receptor knockout mice: Novel phenotypes and clinical implications. Ann. Rev. Pharmacol. Toxicol. 2004, 44, 423450.], and orally administrated eggplant-derived ACh may act on it to stimulate the parasympathetic nervous system. This stimulation travels to the medulla oblongata, where sympathetic nervous activity is automatically reduced due to reciprocal innervation.

Abnormal neuromodulation is considered a cause of BP elevation in SHRs. Blood catechol amine level in SHRs was reported to be higher than that in normotensive Wister Kyoto (WKY) rats, which was attributed to abnormal acceleration of sympathetic nervous activity in SHRs [Tsunoda, M.; Takezawa, K.; Ina, Y.; Nagashima, K.; Ohomori, K.; Kobayashi, S.; Imai, K. New approach for measurement of sympathetic nervous abnormality in conscious, spontaneously hypertensive rats. Jpn. J. Pharmacol. 2000, 83, 3945, Donohue, S. J.; Stitzel, R. E.; Head, R. J. Time course of changes in the norepinephrine content of tissues from spontaneously hypertensive and Wistar Kyoto rats. J. Pharmacol. Exp. Ther. 1988, 245, 24-31.]. The sensitivity of the baroreceptor reflex function in SHRs was also reported to be lower than that in WKY rats [Kumagai, H.; Averill, D. B.; Ferrario, C. M. Renal nerve activity in rats with spontaneous hypertension: Effect of converting enzyme inhibitor. Am. J. Physiol. 1992, 263, 109-115.]. It was suggested that neuromodulation to control BP varied among BP categories, and ACh in eggplant reduced BP more efficiently in participants with grade 1 hypertension versus those with normal high BP. Regarding the primary outcome, hospital BP, eggplant intake improved only DBP in normal-high BP participants, but it improved both SBP and DBP in grade 1 hypertension participants. The difference in BP between the eggplant and placebo groups was 5.6 mmHg for DBP in the normal-high BP participants, and 7.6 mmHg for SBP and 7.3 mmHg for DBP in the grade 1 hypertension participants.

Stress and negative PSs increase sympathetic nervous activity; thus, suppression of sympathetic nervous activity could reduce stress and improve PSs. Therefore, participants with stress were employed in the study to evaluate the effect of eggplant on stress and PSs using VAS and POMS-2 questionnaires, respectively. A very simple VAS questionnaire was used to evaluate stress after oral intake tests in another work [Diaper, A.; Papadopoulos, A.; Rich, A. S.; Dawson, G. R; Dourish, C. T.; Nutt, D. J.; Bailey, J. E. The effect of a clinically effective and non-effective dose of lorazepam on 7.5% C02-induced anxiety. Hum. Psychopharmacol. 2012, 27, 540-548.]. PSs are reported to be involved in the development and state of hypertension [Andrew, M. J.; Baker, R. A.; Kneebone, A. C.; Knight, J. L. Mood state as a predictor of neuropsychological deficits following cardiac surgery. J. Psychosom. Res. 2000, 48, 537-546, Hassan, K.; Elimeleh, Y.; Shehadeh, M.; Fadi, H.; Rubinchik, I. The relationship between hydration status, male sexual dysfunction and depression in hemodialysis patients. Ther. Clin. Risk Manag. 2018, 14, 523-529.], and the POMS-2 test was therefore used to assess PSs [Yu, B. H.; Dimsdale, J. E.; Mills, P. J. Psychological states and lymphocyte beta-adrenergic receptor responsiveness. Neuropsychopharmacology 1999, 21, 147-152.].

Results of the VAS and POMS-2 tests indicated that long-term intake of eggplant powder had no effect on stress but improved PSs. The antistress effect of eggplant should be further examined with an objective method instead of the subjective assessment with the VAS questionnaire. In the POMS-2 test, eggplant intake improved negative PSs (‘confusion-bewilderment’ of all participants and ‘depression-dejection’; ‘confusion-bewilderment’, ‘anger-hostility’, and ‘total mood disturbance’) in the normal-high BP group and positive PSs (‘friendliness’ and ‘vigor-activity’) in the grade 1 hypertension group. The results of the POMS-2 test also indicated different efficiency of eggplant for participants indifferent BP categories. The trend of improvement in the PS from week 0 to week 4 was suggested to bean effect of acclimatization. At week 8, PSs seemed to deteriorate; this may be because of an earthquake that occurred on September 6, 15 days prior to the week 8 examination, although there was no impact on the test overall.

Dietary intervention in this study included the intake of eggplant powder capsules and avoidance of daily ACh-rich foods, including eggplant and bamboo shoot. The daily dose of eggplant powder in the treatment group capsules was 1.2 g, which contained 4.39 kcal. The placebo capsules also contained 4.35 kcal, without the eggplant powder. Therefore, we concluded that the observed BP- and PS-improving effects were due to the test food. In addition to ACh, eggplant contains GABA [Horie, H.; Ando, S.; Saito, T. The contents of γ-amino butyric acid in eggplant and its accumulation with heat treatment. J. Jpn. Soc. Food Sci. 2013, 60, 661-664.] and CA [Singh, A. P.; Luthria, D.; Wilson, T.; Vorsa, N.; Singh, V.; Banuelos, G. S.; Pasakdee, S. Polyphenols content and antioxidant capacity of eggplant pulp. Food Chem. 2009, 114, 955-961.], which are compounds with hypotensive effect. The effective doses of GABA and CA (derived from coffee) for antihypertensive effect in adult individuals are 12.3 mg/day and 271 mg/day, respectively, according to the “Foods with Function Claims” in Japan. The test food in this study contained 7.65 mg/day of GABA (62.20% of the effective dose) and 12.3 mg/day of CA (4.54% of the effective dose), which were both lower than the respective effective doses. We have reported that eggplant-derived ACh, not GABA or CA, is the main compound responsible for the hypotensive effects of eggplant in SHRs [Non patent literature 16].

This result should be applied when considering the active ingredient in eggplant to reduce BP in a clinical trial of participants with relatively higher blood pressure. Both GABA and CA in the test food were estimated to be ineffective alone because of the low dose. No data indicating a synergistic effect enhancing the individual hypotensive effects have been reported. It is reasonable to assume that the antihypertensive effect was from the ACh. Therefore, the BP-improving effects of GABA and CA in the test food should be limited in this study, and the main antihypertensive component in the eggplant powder was concluded to be ACh. A study on the antihypertensive effect of orally administered ACh is currently being conducted, and we expect positive results. This randomized controlled trial confirmed the safety of eggplant powder at a dose equivalent to 22 g/day of raw eggplant.

In conclusion, eggplant powder intake significantly improved BP and PSs, compared with those in the control group. Changes in hospital and home BPs overall and in the normal-high BP group were transiently improved at week 4 and 8. The reasons were estimated to be environmental factors. It is well-known that BP, including hospital and home BP, varies by the season [Lewington, S.; Li, L.; Sherliker, P.; Guo, Y.; Millwood, L.; Bian, Z.; Whitlock, G.; Yang, L.; Collins, R; Chen, J.; et al. Seasonal variation in blood pressure and its relationship with outdoor temperature in 10 diverse regions of China: The China Kadoorie Biobank. J. Hypertens. 2012, 30, 1383-1391.]. From summer to winter, BP increases with decreasing temperature. The clinical trial was conducted from the end of summer to early winter, and the average temperature dropped from approximately 27.9° C. to 9.6° C., corresponding with an estimated 3.5 mmHg increase in BP.

The increase in BP due to temperature decrease and the BP-lowering effect of eggplant powder intake could be mediated through neuromodulation, and both effects may have competed against each other, thus attenuating the effect of eggplant after 4 or 8 weeks from beginning of the test. The measured hospital BPs overall and in the grade 1 hypertension group were also transiently improved at week 12, which should be caused by prolonged intake of eggplant powder. These characteristic effects of eggplant powder seemed to be mild and gradual, which was different from the effects of current antihypertensives. Therefore, continuous intake of eggplant is expected to improve BP in a person with normal-high BP or grade 1 hypertension. Eggplant is recognized as safe. This clinical trial confirmed the safety as well as the BP and PSs improving effects of eggplant powder. These novel effects were expected to support the use of the ACh-containing eggplant powder as a safe treatment for BP and mental health.

### 3.4. Conclusions

A randomized, double-blind, placebo-controlled, parallel-group comparative study showed that ingestion of eggplant powder for 12 weeks improved BP and PSs without side effect. The eggplant powder dose was 1.2 g/day contained 2.3 mg of eggplant-derived choline ester (ACh), which was a novel functional food factor. This was the first evidence of the antihypertensive and PS-improving effects of eggplant. Eggplant is expected to be utilized as a safe treatment for BP and mental health.

As described above, the composition according to the present embodiment can provide a new composition having an excellent antihypertensive effect and a psychological state improving effect that can be easily and inexpensively produced from the fruit of eggplant (Solanum melongena). In addition, when orally administered, it has a remarkable antihypertensive effect as compared with a composition having an antihypertensive effect containing a conventional choline ester as an active ingredient. This can be used to produce “foods with function claims” or pharmaceuticals for the treatment of hypertension and the like.

The eggplant fruit, which is the raw material of the composition according to the present embodiment, is widely recognized as edible, and the composition of the present invention can be comprised only of eggplant-derived components, is highly safe, and is suitable for ingestion for a long period of time. The composition of the present invention includes a water extract of eggplant fruit, a concentrated extract obtained by concentrating the extract, a dry powder (freeze-dried powder, hot-air dried powder, spray-dried powder, and the like), a suspension of the dry powder, and the like. This could be used for foods and drinks and pharmaceuticals in various forms.

1. A composition for improving a psychological state, which comprises a fruit-derived component of eggplant (Solanum melongena).

2. The composition for improving a psychological state according to claim 1, which comprises a water-soluble component of the fruit of eggplant (Solanum melongena) as an active ingredient.

3. The composition for improving a psychological state according to claim 1, which consists of a water-soluble component of the fruit of eggplant (Solanum melongena).

4. The composition for improving a psychological state according to claim 1,
wherein it is orally ingested at a dose such that an amount of choline ester in the water-soluble component is greater than or equal to 0.5 μg/kg body weight and less than or equal to 50 mg/kg body weight.

5. The composition for improving a psychological state according to claim 1, which has a choline ester content of greater than or equal to 5 μg and less than or equal to 50 mg and is for oral ingestion.

6. The composition for improving a psychological state according to claim 4,
wherein the choline ester is selected from any one of acetylcholine, butylcholine and propionylcholine.

7. The composition for improving a psychological state according to claim 1, which is a dry powder.

8. The composition for improving a psychological state according to claim 1, which is a food composition.

9. A method for producing a composition for improving a psychological state, which comprises squeezing an eggplant (Solanum melongena) fruit to obtain a juice.

10. A method for producing a composition for improving a psychological state containing an eggplant-derived component,
wherein water is added to a fruit of eggplant (Solanum melongena) and the fruit to which the water is added is squeezed to obtain a juice.

11. The method for producing the psychological state improving composition according to claim 10, which comprises crushing the fruit before or after adding the water to the fruit.

12. The method for producing the composition for improving a psychological state according to claim 9, which comprises heating the fruit.

13. The method for producing the composition for improving a psychological state according to claim 9, which comprises drying the juice into a powder.

14. A composition for improving a psychological state produced by the production method according to claim 9.

15. Use of eggplant (Solanum melongena) fruit to improve a psychological state.

16. The use of eggplant fruit according to claim 15 by oral ingestion.

17. A composition for improving a psychological state, which comprises a water-soluble component of a food made from an agricultural product containing a high amount of choline ester as an active ingredient.

18. The composition for improving a psychological state according to claim 5,
wherein the choline ester is selected from any one of acetylcholine, butylcholine and propionylcholine.

19. The method for producing the composition for improving a psychological state according to claim 10, which comprises heating the fruit.

20. The method for producing the composition for improving a psychological state according to claim 10, which comprises drying the juice into a powder.

21. A composition for improving a psychological state produced by the production method according to claim 10.
